SEQUENCE LISTING (2 sequences)

SEQ ID NO: 1 - DNA, 1227 bp, Homo sapiens
atgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctga

SEQ ID NO: 2 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

(19) United States
(12) Patent Application Publication
(10) Pub. No.: US 2022/0226391 A1
(43) Pub. Date: Jul. 21, 2022

(54) COMPOSITIONS AND METHODS FOR THYMIC REGENERATION AND T-CELL RECONSTITUTION

(71) Applicant: Angiocrine Bioscience, Inc., San Diego, CA (US)

(72) Inventors: Kenneth N. Wills, San Diego, CA (US); Karolina Kucharova, San Diego, CA (US); Michael Daniel Ginsberg, San Diego, CA (US); Daniel Joseph Nolan, Hawthorne, NY (US); Paul William Finnegan, Del Mar, CA (US)

(21) Appl. No.: 17/614,521

(22) PCT Filed: May 28, 2020
(86) PCT No.: PCT/US20/34932

§ 371 Date: Nov. 27, 2021

Related U.S. Application Data
(60) Provisional application No. 62/853,452, filed on May 28, 2019.

(51) Int. Cl. A61K 35/44; C12N 5/071; C12N 15/63

(52) U.S. Cl. CPC A61K 35/44; C12N 15/63; C12N 5/069

(57) ABSTRACT

The present invention provides non-thymic endothelial cells (ntECs) engineered to express adenovirus E4ORF1 and/or BMP4, and compositions comprising such engineered ntECs. The present invention also provides methods of using such ntECs in therapy, for example to enhance thymic regeneration (including T cell reconstitution) in subjects in need thereof. Such subjects include those that have a damaged thymus, defective thymic function, insufficient T-cell output, and/or that are immunocompromised—for example as a result of ageing, infection (e.g. with HIV), treatment with radiation therapy, treatment with chemotherapy, or myeloablative conditioning in preparation for an organ/tissue transplant.

### CROSS-REFERENCE TO RELATED APPLICATIONS

This application claims the benefit of priority of U.S. Provisional Patent Application No. 62/853,452 filed on May 28, 2019.

### SEQUENCE LISTING

The instant application contains a Sequence Listing which has been submitted electronically in ASCII format and is hereby incorporated by reference in its entirety. Said ASCII copy, created on May 28, 2020 is named Angiocrine_024_WO1_SL.txt and is 5,546 bytes in size.

### INCORPORATION BY REFERENCE

For the purpose of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Many of the general teachings provided in U.S. Pat. No. 8,465,732 can be used in conjunction with the present invention, or can be adapted for use with the present invention. Accordingly, the entire contents of U.S. Pat. No. 8,465,732 are hereby expressly incorporated by reference into the present application.

### BACKGROUND

The thymus supports the development of T cells from hematopoietic progenitor cells migrating from the bone marrow. Legrand et al. (2007); “Human thymus regeneration and T cell reconstitution;” Seminars in Immunology; Vol. 19; No. 5; pp 280-288. Thymic activity gradually declines with age resulting in diminished T-cell output and compromised immune function. Id. The thymus is also very sensitive to insult—it is easily damaged leading to depleted T cell output and immune deficiency in a variety of situations including in response to chemotherapy, radiation exposure, conditioning regimens used before organ transplant (such as bone marrow transplantation), and infection (such as HIV infection). Id. Regeneration of thymic tissue can replenish the T cell compartment—restoring immune function. Id. While the thymus does have some intrinsic regenerative capacity, the extent and speed of this thymic regeneration is frequently insufficient, leaving patients severely immuno-compromised and at risk from potentially life-threatening infections. As such, there is a need in the art for compositions and methods that can enhance both thymic regeneration and T-cell reconstitution. The present invention addresses these needs.

### SUMMARY OF THE INVENTION

The present invention is based, in part, upon the surprising discovery that thymic regeneration can be induced in vivo by administering to a living subject non-thymic endothelial cells (“ntECs”) engineered to express either an adenovirus E4ORF1 polypeptide or both an adenovirus E4ORF1 polypeptide and BMP4. As shown in the Examples section of this patent disclosure, administration of engineered ntECs expressing either E4ORF1 alone, or E4ORF1 together with BMP4, to living subjects leads to enhanced thymic regeneration and T-cell reconstitution. These effects are particularly surprising given that prior published studies had demonstrated that, while administration of endothelial cells could enhance thymic regeneration, this effect could only be achieved if thymic endothelial cells were used. See Wertheimer et al., (2018); “Production of BMP4 by endothelial cells is crucial for endogenous thymic regeneration;” Sci. Immunol. 3, 2736. In these prior studies thymic regeneration was not observed when non-thymic endothelial cells were used. Id. The discovery that engineered ntECs expressing E4ORF1 alone, or both BMP4 and E4ORF1, can be used to induce thymic regeneration and T-cell reconstitution in vivo has several important practical implications, one of which is that it eliminates the need to perform complicated and invasive procedures to obtain and culture endothelial cells from a patient's thymus. Instead, endothelial cells for use in thymic regeneration and T-cell reconstitution protocols can be obtained from much more accessible sources—such as from adipose tissue, skin tissue or umbilical cord tissue—greatly simplifying the applicability of endothelial cell therapy for thymic regeneration.

Accordingly, the present invention provides a variety of novel compositions and methods.

In one embodiment the present invention provides a population of engineered ntECs that express BMP4 (i.e. BMP4+ ntECs). In another embodiment, the present invention provides a population of engineered ntECs that express an adenovirus E4ORF1 polypeptide (i.e. E4ORF1+ ntECs). In another embodiment, the present invention provides a population of engineered ntECs that express BMP4 and an adenovirus E4ORF1 polypeptide (i.e. BMP4+E4ORF1+ ntECs). In some embodiments these populations of engineered ntECs are isolated cell populations. In some embodiments the populations of engineered ntECs are substantially pure cell populations. In some embodiments the populations of engineered ntECs are present in vitro, for example in cell culture. In some embodiments the populations of engineered ntECs are present ex vivo. In some embodiments the populations of engineered ntECs are present in vivo. In some embodiments the populations of engineered ntECs are present in a composition, such as a therapeutic composition suitable for administration to a living subject. For example, in some embodiments the present invention provides a therapeutic composition comprising a population of engineered ntECs and a physiological saline suitable for administration to a living subject. Similarly, in some embodiments the present invention provides a therapeutic composition comprising a population of engineered ntECs and a biocompatible matrix material, such as a liquid biocompatible matrix material (e.g. Matrigel) or a solid biocompatible matrix material.

In some embodiments the engineered ntECs provided herein, and/or compositions comprising these engineered ntECS, may be used in various therapeutic applications, including, methods for enhancing thymic regeneration and/or T-cell reconstitution in a living subject, such as a subject that has a deficiency in thymic tissue mass, thymic function, or T-cell production, or that is otherwise immunocompromised. In some embodiments such subjects are of advanced age, have a viral infection (e.g. an HIV infection), have been exposed to radiation (e.g. radiation therapy), have been treated with chemotherapy, or have been treated with myeloablative conditioning agents—for example in preparation for an organ transplant such as a bone marrow or hematopoietic stem cell transplant (HSCT).

For example, in some embodiments the present invention provides a method for enhancing thymic regeneration and/or T-cell reconstitution in a subject, the method comprising administering to a subject in need thereof an effective amount of ntECs engineered to express E4ORF1 or both E4ORF1 and BMP4, or a therapeutic composition comprising such ntECs, thereby stimulating thymic regeneration and/or T-cell reconstitution in the subject.

The endothelial cells (ECs) can be from any non-thymic source. Examples of suitable sources of the ECs (ntECs) include, but are not limited to, adipose tissue (i.e. adipose ECs), skin tissue (i.e. skin ECs), cardiac tissue (i.e. cardiac ECs), kidney tissue (i.e. kidney ECs), lung tissue (i.e. lung ECs), liver tissue (i.e. liver ECs), bone marrow tissue (i.e. bone marrow ECs), umbilical vein (i.e. umbilical vein ECs—or “UVECs”), and the like.

In some embodiments the ntECs are adult ECs. In some embodiments the ntECs are juvenile ECs. In some embodiments the ntECs are fetal ECs. In some embodiments the ntECs are embryonic ECs. In some embodiments the ntECs are differentiated ECs. In some embodiments the ntECs are derived from endothelial progenitor cells. In some embodiments the ntECs are derived from stem cells. In some embodiments the ntECs are from primary tissue cultures. In some embodiments the ntECs are ECs of an endothelial cell line. In cases where the ntECS are to be used in a treatment method as described herein, in some embodiments the ntECs are autologous to the subject to whom the cells are to be administered, while in other embodiments the ntECs are allogeneic to the subject to whom the cells are to be administered.

In some embodiments the ntECs comprise a recombinant nucleic acid molecule that comprises a nucleotide sequence that encodes the recited molecule or molecules—for example a nucleotide sequence that encodes BMP4 and/or a nucleotide sequence that encodes an adenovirus E4ORF1 polypeptide. In embodiments where both BMP4 and E4ORF1 are used, the nucleotide sequence that encodes the BMP4 and the nucleotide sequence that encodes the E4ORF1 polypeptide may be provided in the same recombinant nucleic acid molecule or in different recombinant nucleic acid molecules. In some embodiments the nucleotide sequence that encodes the BMP4 and/or the nucleotide sequence that encodes the E4ORF1 polypeptide is operatively linked to a heterologous promoter. In some embodiments the recombinant nucleic acid molecule is a plasmid vector. In some embodiments the recombinant nucleic acid molecule is an expression vector. In some embodiments the recombinant nucleic acid molecule is a viral vector, such as, for example, a lentiviral vector. In some embodiments the nucleotide sequence that encodes the E4ORF1 and/or BMP4 polypeptide is transiently expressed in the ntECs. In some embodiments the nucleotide sequence that encodes the BMP4 and/or E4ORF1 polypeptide is stably expressed in the ntECs. In some embodiments the nucleotide sequence that encodes the E4ORF1 and/or BMP4 polypeptide is integrated into the genome of the ntECs.

In some embodiments, where a recombinant nucleic acid molecule that comprises a nucleotide sequence that encodes an E4ORF1 polypeptide is used, the recombinant nucleic acid molecule is not an adenovirus genome. In some embodiments, where a recombinant nucleic acid molecule that comprises a nucleotide sequence that encodes an E4ORF1 polypeptide is used, the recombinant nucleic acid molecule is not a naturally occurring adenovirus genome. In some embodiments, where a recombinant nucleic acid molecule that comprises a nucleotide sequence that encodes an E4ORF1 polypeptide is used, the recombinant nucleic acid molecule is not an adenoviral vector.

These and other embodiments of the invention are described further in other sections of this patent disclosure. In addition, as will be apparent to those of skill in the art, certain modifications and combinations of the various embodiments described herein fall within the scope of the present invention.

### BRIEF DESCRIPTION OF THE DRAWINGS

FIG. 1. In vitro expansion assay of 4 cell lines derived from same umbilical cord. At the first day, a small flask with 25 cm2 surface area was seeded with 500,000 cells of each cell line. All cell lines during expansion grown in the presence of serum. While E4ORF1+ HUVEC and BMP4+E4ORF1+ HUVEC covered in less than 15 days 4 flask with surface area 225 cm2 (4×T225), HUVEC and BMP4+ HUVEC covered only 1 T225 flask (1×T225) in 16 days.

FIG. 2A-C. BMP4+E4ORF1+ ntECs stimulate thymic regeneration. Results from experiments described in Example. 2. Each graph shows the numbers of live thymic cells (FIG. 2. A) live medullar epithelial cells (mTEC) (FIG. 2B), and recovered live cortical epithelial cells (cTEC) (FIG. 2C) for the following treatment groups: (1) NoRad, No ECs, (2) Rad. No ECs, (3) Rad, Mu Thymic ECs, (4) Rad Mu BMEC, and (5) Rad. Mu BMEC/BMP4. The y axis shows the number of total cells isolated from the thymus of each individual animal. Significant differences between treatment groups are indicated by asterisks where * indicates a P-value <0.05, ** indicates a P-value <0.01, and *** indicates a P-value <0.001. Mu=murine (i.e. mouse). BMECs are bone marrow endothelial cells. Mu BMEC/BMP4 cells express both E4ORF1 and BMP4.

FIG. 3A-F. BMP4+E4ORF1+ ntECs stimulate thymic regeneration. Results from experiments described in Example. 2. The graphs show the number of cells of the indicated types isolated from the thymus of each individual animal. FIG. 3A—overall thymic epithelial content measured by number of CD45− EpCam+ cells. FIG. 3B—actively proliferating thymic epithelial cell content measured by number of CD45− EpCam+ Ki67+ cells. FIG. 3C—thymic epithelial progenitor cell (TEPC) content measured by number of EpCam+ alpha6 integrin+ Sca1+ cells. FIG. 3D—actively proliferating TPEC content measured by number of EpCam+ alpha6 integrin+ Sca1+ Ki67+ cells. FIG. 3E—thymic epithelial progenitor cell (TEPC) content measured by number of CD45− K5+ K8+ cells. FIG. 3F—actively proliferating TEPC content measured by number of CD45− K5+ K8+ Ki67+ cells. In each case data from two treatment groups is shown. A no EC control (“650 cGy No ECs”) and a group treated with BMP4+E4ORF1+ human umbilical vein endothelial cells (HUVECs) (“650 cGy 500K AB245 cells”). Significant differences between control and treatment groups are indicated by their P values.

FIG. 4. Total thymic cellularity 3 weeks after transplant. Total thymic cellularity 3 weeks (3 W) after lethal (100 cGy) total body irradiation and cell transplant. The co-infusion of rescue murine bone marrow (marrow-only) with BMP4+ E4ORF1+ HUVEC (E4/BMP4) tended to give a numerically greater effect on recovery of thymic cells than their E4ORF1+ HUVEC (E4), non-BMP4 expressing parent. The statistical significance between groups was tested with Kruskal Wallis One Way ANOVA and Dunn's multiple comparisons tests when the Marrow-only group was compared with other irradiated groups, i.e. groups treated with human cells.

FIG. 5. Number of donor CD45+ cells in the thymus. See Example 5 for further description of data in this figure.

FIG. 6A-B. Recovery of CD3⁺ cells in the thymus. See Example 5 for further description of data in this figure.

FIG. 7A-B. Recovery of T lymphocytes expressing either CD4 or CD8. See Example 5 for further description of data in this figure.

FIG. 8A-B. Relative contribution of CD4+ and CD8+ cells to total CD3+ cells. See Example 5 for further description of data in this figure.

FIG. 9. T-Lymphocyte maturation—schematic representation. See Example 5 for further description of information in this figure.

FIG. 10 A-B. Number (FIG. 10A) and percentage (FIG. 10B) of live CD3+ donor Double positive (DP) T-cells 3 weeks after 1000 cGY TBI. See Example 5 for further description of data in this figure.

FIG. 11 A-B. Number (FIG. 11A) and percentage (FIG. 11B) of live Double Negative (DN) donor CD45+ cells 3 weeks after 1000 cGY TBI. See Example 5 for further description of data in this figure.

FIG. 12 A-D. Number of DN subpopulation cells in recovering thymus. FIG. 12A—DN1 cells. FIG. 12B—DN2 cells. FIG. 12C—DN3 cells. FIG. 12D—DN4 cells. See Example 5 for further description of data in this figure.

FIG. 13 A-D. Distribution of DN subpopulations within DN CD3 cells. FIG. 13A—DN1 cells. FIG. 13B—DN2 cells. FIG. 13C—DN3 cells. FIG. 13D—DN4 cells. See Example 5 for further description of data in this figure.

FIG. 14. Number of live CD45 negative/EpCam+ cells 3 weeks after 1000 cGY TBI. See Example 5 for further description of data in this figure.

FIG. 15. Recovery of EpCAM+/Sca1+ cells. See Example 5 for further description of data in this figure.

FIG. 16. Recovery of EpCAM+/Sca1+/α6+ cells. See Example 5 for further description of data in this figure.

FIG. 17. Recovery of EpCAM+ cortical TECs (cTECs). See Example 5 for further description of data in this figure.

FIG. 18. Recovery of Proliferating (Ki67+) cTECs. See Example 5 for further description of data in this figure.

FIG. 19. Recovery of medullary TECs (mTECs). See Example 5 for further description of data in this figure.

FIG. 20. Recovery of proliferating mTECs. See Example 5 for further description of data in this figure.

FIG. 21. BMP4+ E4ORF1+ ntECs Enhance Survival Following Total Body Irradiation and Bone Marrow Transplant. Percentage survival (y axis) is plotted against time in days (x axis) for the indicated treatment groups. The ECs are BMP4+ E4ORF1+ human umbilical vein endothelial cells (HUVECs). Animal survival 5 weeks after lethal total body irradiation is shown. Animals received 200,000 or 500,000 rescue murine bone marrow dose (BM) with/without BMP4+ E4ORF1+ HUVEC.

### DETAILED DESCRIPTION

The “Summary of the Invention,” “Figures,” “Brief Description of the Figures,” “Examples,” and “Claims” sections of this patent disclosure describe some of the main embodiments of the invention. This “Detailed Description” section provides certain additional description relating to the compositions and methods of the present invention, which is intended to be read in conjunction with all other sections of this patent disclosure. Furthermore, and as will be apparent to those in the art, the different embodiments described throughout this patent disclosure can be, and are intended to be, combined in various ways. Such combinations of the specific embodiments described herein are intended to fall within the scope of the present invention

Certain definitions and abbreviations are provided below. Other terms or phrases may be defined elsewhere in this patent disclosure or may have meanings that are clear from the context in which they are used. Unless defined otherwise herein, or unless some other meaning is clear from their use in context herein, all technical and scientific terms and abbreviations used herein have the same meaning as commonly understood by one of ordinary skill in the art to which this invention is related. For example, The Dictionary of Cell and Molecular Biology (5th ed. J. M. Lackie ed., 2013), the Oxford Dictionary of Biochemistry and Molecular Biology (2d ed. R. Cammack et al. eds., 2008), and The Concise Dictionary of Biomedicine and Molecular Biology (2d ed. P-S. Juo, 2002) can provide one of skill with general definitions of some terms used herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10.

Wherever embodiments are described with the language “comprising,” otherwise analogous embodiments described in terms of “consisting of” and/or “consisting essentially of” are included.

As used herein, the terms “about” and “approximately,” when used in relation to numerical values, mean within + or −10% of the stated value.

As used herein, the term “allogeneic” means deriving from, originating in, or being members of the same species, where the members are genetically related or genetically unrelated but genetically similar. In embodiments involving administration of allogeneic ntECs to a subject, the allogeneic cells are obtained from a donor of the same species as the subject to whom the cell will be administered (i.e. the recipient). In some embodiments the allogeneic cells are obtained from a donor having the same MHC/HLA type as the subject to whom the cells will be administered (i.e. the recipient)—i.e. the donor of the cells and the recipient of the cells are MHC-matched or HLA-matched. In some embodiments, cells (e.g. ntECs) are: (a) obtained from a donor, (b) maintained and/or cultured and/or expanded and/or genetically modified ex vivo, and (c) subsequently administered into a subject of the same species as the donor. For example, in some embodiments, ntECs are obtained from a donor, genetically modified ex vivo to render them BMP4+ and/or E4ORF1+, and then administered to a recipient subject of the same species as the donor. Similarly, in some embodiments, ntECs are obtained from a donor, genetically modified ex vivo to render them BMP4+ and/or E4ORF1+, and then administered to a recipient subject of the same species and same MHC/HLA type as the donor.

As used herein, the term “autologous” means deriving from or originating in the same subject. In embodiments involving administration of autologous ntECs to a subject, the autologous ntECs are obtained from the subject to whom the ntECs will be administered (i.e. the donor and recipient of the ntECs are the same individual). In some embodiments, cells (e.g. ntECs) are: (a) obtained from a subject, (b) maintained and/or cultured and/or expanded and/or genetically modified ex vivo, and (c) subsequently administered to the same subject. For example, in some such embodiments, ntECs are obtained from a subject, genetically modified ex vivo to render them BMP4+ and/or E4ORF1+, and then administered to the same subject.

As used herein, the abbreviation “BMP4” refers to bone morphogenetic protein 4 or a nucleotide sequence that encodes that protein—as will be clear from the context of use.

As used herein, the abbreviation “EC(s)” refers to an endothelial cell(s). As used herein the abbreviation “ntEC(s)” refers to non-thymic endothelial cell(s).

As used herein, the abbreviation “E4ORF1” refers to open reading frame (ORF) 1 of the early 4 (E4) region of an adenovirus genome, or a polypeptide/protein encoded by that ORF—as will be clear from the context of use.

The term “culturing” as used herein, refers to the propagation of cells on or in media of various kinds. “Co-culturing” refers to the propagation of two or more distinct types of cells on or in media of various kinds.

As used herein the term “effective amount” refers to an amount of ntECs, or a therapeutic composition comprising ntECs, that is sufficient to achieve the stated treatment outcome to a detectable level—for example as assessed using one or the methods described in the Examples section of this patent application for measuring treatment outcomes. Treatment outcomes are described further below (see “treatment” definition—which refers to various parameters/treatment outcomes). An appropriate “effective amount” in any individual case may be determined empirically, for example using standard techniques known in the art, such as dose escalation studies, and may be determined taking into account such factors as the planned route of administration, desired frequency of administration, etc. Furthermore, an “effective amount” may be determined using assays such as those described in the Examples section of this patent disclosure. In some embodiments an effective amount is about 5×10⁶ ntECs per kg of the subject's body weight. In some embodiments an effective amount is from about 1×10⁶ to about 50×10⁶ ntECs per kg of the subject's body weight. In some embodiments an effective amount is from about 5×10⁶ to about 25×10⁶ ntECs per kg of the subject's body weight. In some embodiments an effective amount is about 5×10⁶ ntECs per kg of the subject's body weight. In some embodiments an effective amount is about 10×10⁶ ntECs per kg of the subject's body weight. In some embodiments an effective amount is about 15×10⁶ ntECs per kg of the subject's body weight. In some embodiments an effective amount is about 20×10⁶ ntECs per kg of the subject's body weight. In some embodiments an effective amount is about 25×10⁶ ntECs per kg of the subject's body weight. In some embodiments an effective amount is about 30×10⁶ ntECs per kg of the subject's body weight. In some embodiments an effective amount is about 35×10⁶ ntECs per kg of the subject's body weight. In some embodiments an effective amount is about 40×10⁶ ntECs per kg of the subject's body weight. In some embodiments an effective amount is about 45×10⁶ ntECs per kg of the subject's body weight. In some embodiments an effective amount is about 50×10⁶ ntECs per kg of the subject's body weight.

The term “engineered” when used in relation to non-thymic endothelial cells (ntECs) refers to ntECS cells that have been engineered by man to result in the recited phenotype (e.g. E4ORF1 expression, BMP4 expression, or E4ORF1 and BMP4 expression), or to express a recited nucleic acid molecule or polypeptide. The term “engineered cells” is not intended to encompass naturally occurring cells, but is, instead, intended to encompass, for example, cells that comprise a recombinant nucleic acid molecule, or cells that have otherwise been altered artificially (e.g. by genetic modification), for example so that they express a polypeptide that they would not otherwise express, or so that they express a polypeptide at substantially higher levels than that observed in non-engineered endothelial cells (e.g. so that they over express BMP4).

The terms “genetic modification” and/or “genetically modified” and/or “gene-modified” refer to any addition, deletion, alteration or disruption of or to a nucleotide sequence or to a cell's genome or to a cell's content of genetic material. In some embodiments, the endothelial cells described herein may, in addition to being genetically modified to provide a nucleic acid molecule that encodes E4ORF1 and/or a nucleic acid molecule that encodes BMP4, may also comprise one or more other genetic modifications—as desired. The term “genetic modification” and the above related terms encompass both transient and stable genetic modification and encompass the use of various different gene delivery vehicles and methods including, but not limited to, transduction (viral mediated transfer of nucleic acid to a recipient, either in vivo or in vitro), transfection (uptake by cells of isolated nucleic acid), liposome mediated transfer and others means of gene delivery that are well known in the art.

As used herein the term “isolated” refers to a population of cells that is separated from at least one other cell population, product, compound, or composition with which it is associated in its usual state, and/or refers to a population of cells that are not in the body of a living subject.

As used herein, the term “recombinant” refers to nucleic acid molecules that are isolated, generated and/or designed by man (including by a machine) using methods of molecular biology and genetic engineering (such as molecular cloning), and that either comprise nucleotide sequences that do not exist in nature, or are comprised within nucleotide sequences that do not exist in nature, or are provided in association with nucleotide sequences that they would not be associated with in nature, or that are provided in the absence of nucleotide sequences with which they would ordinarily be associated in nature. Thus, recombinant nucleic acid molecules are to be distinguished from nucleic acid molecules that exist in nature—for example in the genome of an organism. For example, a nucleic acid molecule that comprises a complementary DNA or “cDNA” copy of an mRNA sequence, without any intervening intronic sequences such as would be found in the corresponding genomic DNA sequence, would thus be considered a recombinant nucleic acid molecule. By way of a further example, a recombinant E4ORF1 nucleic acid molecule might comprise an E4ORF1 coding sequence operatively linked to a promoter and/or other genetic elements with which that coding sequence is not ordinarily associated in a naturally-occurring adenovirus genome. Similarly, a recombinant BMP4 nucleic acid molecule might comprise BMP4-coding sequences operatively linked to a promoter and/or other genetic elements with which that coding sequence is not ordinarily associated in the genome of an organism.

The term “subject” includes mammals—such as humans and non-human primates, as well as other mammalian species including rabbits, rats, mice, cats, dogs, horses, cows, sheep, goats, pigs and the like. In some embodiments the subjects are mammalian subjects. In some embodiments the subjects are humans. In some embodiments the subjects are non-human primates.

The phrase “substantially pure” as used herein in relation to a cell population refers to a population of cells of a specified type (e.g. as determined by expression of one or more specified cell markers, morphological characteristics, or functional characteristics), that is at least about 50%, preferably at least about 75%, more preferably at least about 85%, and most preferably at least about 95% of the cells making up the total cell population. Thus, a “substantially pure cell population” refers to a population of cells that contain fewer than about 50%, preferably fewer than about 25%, more preferably fewer than about 15%, and most preferably fewer than about 5% of cells that are not of the specified type or types.

The terms “treating” and “regenerating” (and grammatical variations thereof), as well as the associated method terms (e.g. “methods of treating” and “methods of regenerating”), as used herein in relation to the thymus, thymic cells or conditions affecting the thymus refer to increasing or accelerating, or methods that increase or accelerate (collectively “enhance”)—to a detectable degree—either a specified parameter or one or more of the following parameters (a) to (t), each of which has been shown herein to be increased or accelerated (i.e. enhanced) when the engineered ntECs of the invention are administered to living subjects: (a) number of or proliferation of total thymic cells (CD45+ thymocytes and CD45− thymic stromal cells, (b) thymic mass, (c) output of self-restricted and/or self-tolerant and/or immunocompetent and/or naïve T cells, (d) thymic function (such as the support for lymphoid cells such as T cells), (e) number of or proliferation of T cell progenitors, immature T cells or mature T cells, (f) number of or proliferation of CD45⁺ cells, (g) number of or proliferation of CD3⁺ cells, (h) number of or proliferation of CD3⁺CD4⁺ cells, (i) number of or proliferation of CD3⁺CD8⁺ cells, (j) number of or proliferation of CD3⁺CD4⁺ CD8⁺ (double-positive or “DP” cells), (k) number of or proliferation of CD4⁻ CD8⁻ cells (double-negative or “DN” cells, such as DN1, DN2, DN3, and/or DN4 cells), (1) number of or proliferation of thymic stromal cells, (m) number of or proliferation of thymic epithelial cells, (n) number of or proliferation of thymic CD45⁻ EpCAM⁺ cells, (o) number of or proliferation of thymic CD45⁻ EpCAM⁺ Sca1+ cells (TEPC), (p) number of or proliferation of thymic CD45⁻ EpCAM⁺ cells (cTEC), (q) number of or proliferation of thymic CD45⁻ EpCAM⁺ Ki67⁺ (proliferating cTEC cells), (r) number of or proliferation of thymic CD45⁻ EpCAM⁺ cells (medullary thymic epithelial cells (mTEC), (s) number of or proliferation of thymic CD45⁻ EpCAM⁺ Ki67⁺ (proliferating mTEC cells), and (t) number of or proliferation of one or more of the cell types listed in Table A (see below). In certain embodiments, a subject is successfully “treated,” or successful “regeneration” or “recovery” of the thymus or a specified thymic cell type or types is achieved, if there is a permanent or transient increase or acceleration (collectively “enhancement”) of one or more of these parameters/treatment outcomes. In some embodiments the detection of, and/or determination of the amount/level of enhancement (increase, or acceleration) of such parameters/treatment outcomes is assessed in comparison to a suitable baseline or a suitable control—such as in comparison to the level of the parameter before the treatment method is commenced, or in comparison to the level of the parameter in a comparable control subject in which the method is not performed, or in comparison to the level of the parameter if the method is performed in the absence of ntECS (e.g. using a delivery vehicle but not ntECs), or in comparison to the level of the parameter if the method is performed using non-engineered ntECs (e.g. ntECs not expressing BMP4 and/or not expressing E4ORF1). In some embodiments the amount of enhancement (increase or acceleration) of the one or more parameters/treatment outcomes may be any detectable amount. In some embodiments the amount of enhancement (increase or acceleration) of the one or more parameters/treatment outcomes may be any statistically significant amount. In some embodiments the amount of enhancement (increase or acceleration) of the one or more of parameters may be about 10%, 20%, 30%, 40%, 50%, 60%, 70%, 80%, 90%, 100%, 150%, 200%, 250%, 300%, 400%, 500%, or more, as compared to a suitable baseline or suitable control. In some embodiments the amount of enhancement (increase or acceleration) of the one or more of parameters may be such that the parameter/treatment outcome is at about the “normal” level for the subject—i.e. the level that would be expected in a healthy individual, or the level that would be expected in the subject without any illness, or the level that the subject had prior to becoming ill or prior to being treated with chemotherapy, radiation therapy, a pre-transplantation conditioning regimen, or a myeloablative conditioning regimen. In some embodiments the amount of enhancement (increase or acceleration) of the one or more of parameters may be such that the parameter is at a level that is greater than the “normal” level. In some embodiments the amount of enhancement (increase or acceleration) of the one or more of parameters may be such that the parameter is about 50%, or more preferably about 60%, or more preferably about 70%, or more preferably about 80%, or more preferably about 90% of the “normal” level. It should be noted that, for each embodiment of the invention that refers to a method of enhancing thymic regeneration, the corresponding method of increasing thymic regeneration and the corresponding method of accelerating thymic regeneration is also contemplated. Similarly, it should be noted that for each embodiment of the invention that refers to a method of enhancing thymic regeneration, a method of enhancing any one or more of the specific parameters/treatment outcomes listed above (e.g. parameters a-s) is also contemplated.

As used herein the term “thymic cells” refers to cells that ordinarily form part of, or are located in, the thymus, including, but not limited to, those listed in Table A, below. Within the general category of thymic cells are both thymic stromal cells and cells of hematopoietic origin (such as T cell progenitors, immature T cells, and mature T cells) that reside within the thymus for at least some period of time—for example as part of the T cell maturation and T cell education processes that occur within the thymus. The term “thymic regeneration” (defined above) includes both regeneration of thymic stromal cells and regeneration of cells of hematopoietic origin and includes the term “T cell reconstitution.” The effects on cells of hematopoietic origin that occur as part of the thymic regeneration process can be observed based on assessing the cells of hematopoietic origin residing within the thymus and/or by assessing such cells in the circulation—e.g. an enhancement of T cell output from the thymus following administration of the engineered ntECs of the invention can be detected by assessing such cells in the circulation.

TABLE A

| Thymic Cells | Cell Marker Profile | Cell Type |
|---|---|---|
| Hematopoietic | CD45+ | T cell progenitors and T cells (mature |
| Lineage Cells | | and immature) |
| | CD45+ CD3+ | T cells (mature) |
| | CD45+ CD3+ CD4+ | CD4+ T cells (mature) |
| | CD45+ CD3+ CD8+ | CD8+ T cells (mature) |
| | CD45+ CD3+ CD4+ CD8+ | Double-positive T cells (DP) (mature) |
| | CD45+ CD4− CD8− | Double-negative T cells (DN) |
| | | (immature) |
| | CD45+ CD4− CD8− CD44+ CD25− | Double-negative type 1 cells (DN1) |
| | | (immature) |
| | CD45+ CD4− CD8− CD44+ CD25+ | Double-negative type 2 cells (DN2) |
| | | (immature) |
| | CD45+ CD4− CD8− CD44− CD25+ | Double-negative type 2 cells (DN3) |
| | | (immature) |
| | CD45+ CD4− CD8− CD44− CD25− | Double-negative type cells (DN4) |
| | | (immature) |
| Epithelial | CD45−EpCAM+ Sca1+ (α6+ or α6−) | Thymic epithelial progenitor cells |
| Cells | | (TEPC) |
| | CD45−EpCAM+ Sca1+ (α6+ or α6−) | Proliferating thymic epithelial |
| | Ki67+ | progenitor cells (TEPC) |
| | CD45−EpCAM+ | Thymic epithelial cells (TEC) |
| | CD45−EpCAM+ Ki67+ | Proliferating thymic epithelial cells |
| | | (TEC) |
| | CD45− EpCam+ K5+ K8+ | Thymic epithelial progenitor cells |
| | | (TEPC) |
| | CD45−EpCAM+ | Cortical thymic epithelial cells |
| | MHCII⁺Ly51⁺UEA1^{lo} | (cTEC) |
| | CD45−EpCAM+ Ki67+ | Proliferating cortical thymic epithelial |
| | MHCII⁺Ly51⁺UEA1^{lo} | cells (cTEC) |
| | CD45− | Medullary thymic epithelial cells |
| | EpCAM+MHCII⁺Ly51^{lo}UEA1⁺ | (mTEC) |
| | CD45−EpCAM+ Ki67+ | Proliferating medullary thymic |
| | MHCII⁺Ly51^{lo}UEA1⁺ | epithelial cells (mTEC) |

### Nucleic Acid Molecules and Polypeptides

Several of the embodiments of the present invention described herein involve engineered endothelial cells (ntECs) that are E4ORF1+, BMP4+, and/or E4ORF1+ BMP4+—i.e. cells that express an E4ORF1 polypeptide, a BMP4 polypeptide, or both an E4ORF1 polypeptide and a BMP4 polypeptide. These polypeptides are referred to collectively herein as “polypeptides of the invention.”

The “polypeptides of the invention” are encoded by nucleic acid molecules. Thus, in some embodiments the present invention involves nucleic acid molecules that encode an adenovirus E4ORF1 polypeptide, nucleic acid molecules that encode a BMP4 polypeptide, and/or nucleic acid molecules that encode both an adenovirus E4ORF1 polypeptide and a BMP4 polypeptide. Such nucleic acid molecules are referred to collectively herein as “nucleic acid molecules of the invention.”

The polypeptides of the invention and the nucleic acid molecules of the invention may have amino acid sequences or nucleotide sequences that are specified herein or known in the art, or may have amino acid or nucleotide sequences that are variants, derivatives, mutants, or fragments of such amino acid or nucleotide sequences—provided that such a variants, derivatives, mutants, or fragments comprise, or encode, a polypeptide that has, one or more of the functional properties described herein (which include, but are not limited to, an ability to an ability to induce thymic regeneration and/or T-cell reconstitution when expressed in ntECs and administered to subjects in need of thymic regeneration and/or T-cell reconstitution).

In those embodiments involving BMP4 polypeptides, the BMP4 polypeptide may be any mammalian BMP4 polypeptide, such as a human, non-human primate, rabbit, rat, mouse, goat, or pig BMP4 polypeptide. In some embodiments the polypeptide may be a human BMP4 polypeptide. Amino acid sequences of such polypeptides, and nucleic acid sequences that encode such polypeptides, are well known in the art and available in well-known publicly available databases, such as the Genbank database. For example, suitable human amino acid sequences for human BMP4 include those have the following accession numbers: NP_001193.2 GI:157276593 NP_570911.2 GI:157276595, NP_570912.2 GI:157276597, NP_001334841.1 GI:1122781626, NP_001334842.1 GI:1122781519, NP_001334843.1 GI:1122780734, NP_001334844.1 GI:1122781552, NP_001334845.1 GI:1122780682, and NP_001334846.1 GI:1122781077. In the experiments described in the Examples section of this patent disclosure, the human BMP4 polypeptides used was encoded by the nucleotide sequence:

(SEQ ID NO. 1)
ATGATTCCTGGTAACCGAATGCTGATGGTCGTTTTATTATGCCAAGTCCTG
CTAGGAGGCGCGAGCCATGCTAGTTTGATACCTGAGACGGGGAAGAAAAAA
GTCGCCGAGATTCAGGGCCACGCGGGAGGACGCCGCTCAGGGCAGAGCCAT
GAGCTCCTGCGGGACTTCGAGGCGACACTTCTGCAGATGTTTGGGCTGCGC
CGCCGCCCGCAGCCTAGCAAGAGTGCCGTCATTCCGGACTACATGCGGGAT
CTTTACCGGCTTCAGTCTGGGGAGGAGGAGGAAGAGCAGATCCACAGCACT
GGTCTTGAGTATCCTGAGCGCCCGGCCAGCCGGGCCAACACCGTGAGGAGC
TTCCACCACGAAGAACATCTGGAGAACATCCCAGGGACCAGTGAAAACTCT
GCTTTTCGTTTCCTCTTTAACCTCAGCAGCATCCCTGAGAACGAGGTGATC
TCCTCTGCAGAGCTTCGGCTCTTCCGGGAGCAGGTGGACCAGGGCCCTGAT
TGGGAAAGGGGCTTCCACCGTATAAACATTTATGAGGTTATGAAGCCCCCA
GCAGAAGTGGTGCCTGGGCACCTCATCACACGACTACTGGACACGAGACTG
GTCCACCACAATGTGACACGGTGGGAAACTTTTGATGTGAGCCCTGCGGTC
CTTCGCTGGACCCGGGAGAAGCAGCCAAACTATGGGCTAGCCATTGAGGTG
ACTCACCTCCATCAGACTCGGACCCACCAGGGCCAGCATGTCAGGATTAGC
CGATCGTTACCTCAAGGGAGTGGGAATTGGGCCCAGCTCCGGCCCCTCCTG
GTCACCTTTGGCCATGATGGCCGGGGCCATGCCTTGACCCGACGCCGGAGG
GCCAAGCGTAGCCCTAAGCATCACTCACAGCGGGCCAGGAAGAAGAATAAG
AACTGCCGGCGCCACTCGCTCTATGTGGACTTCAGCGATGTGGGCTGGAAT
GACTGGATTGTGGCCCCACCAGGCTACCAGGCCTTCTACTGCCATGGGGAC
TGCCCCTTTCCACTGGCTGACCACCTCAACTCAACCAACCATGCCATTGTG
CAGACCCTGGTCAATTCTGTCAATTCCAGTATCCCCAAAGCCTGTTGTGTG
CCCACTGAACTGAGTGCCATCTCCATGCTGTACCTGGATGAGTATGATAAG
GTGGTACTGAAAAATTATCAGGAGATGGTAGTAGAGGGATGTGGGTGCCGC
TGA.

This nucleotide sequence encodes the following BMP4 amino acid sequence:

(SEQ ID NO. 2)
MIPGNRMLMVVLLCQVLLGGASHASLIPETGKKKVAEIQGHAGGRRSGQSH
ELLRDFEATLLQMFGLRRRPQPSKSAVIPDYMRDLYRLQSGEEEEEQIHST
GLEYPERPASRANTVRSFHHEEHLENIPGTSENSAFRFLFNLSSIPENEVI
SSAELRLFREQVDQGPDWERGFHRINIYEVMKPPAEVVPGHLITRLLDTRL
VHHNVTRWETFDVSPAVLRWTREKQPNYGLAIEVTHLHQTRTHQGQHVRIS
RSLPQGSGNWAQLRPLLVTFGHDGRGHALTRRRRAKRSPKHHSQRARKKNK
NCRRHSLYVDFSDVGWNDWIVAPPGYQAFYCHGDCPFPLADHLNSTNHAIV
QTLVNSVNSSIPKACCVPTELSAISMLYLDEYDKVVLKNYQEMVVEGCGC
R.

In those embodiments involving adenovirus E4ORF1 polypeptides, the polypeptide sequence used may be from any suitable adenovirus type or strain, such as human adenovirus type 2, 3, 5, 7, 9, 11, 12, 14, 34, 35, 46, 50, or 52. In some preferred embodiments the polypeptide sequence used is from human adenovirus type 5. Amino acid sequences of such adenovirus polypeptides, and nucleic acid sequences that encode such polypeptides, are well known in the art and available in well-known publicly available databases, such as the Genbank database. For example, suitable sequences include the following: human adenovirus 9 (Genbank Accession No. CA105991), human adenovirus 7 (Genbank Accession No. AAR89977), human adenovirus 46 (Genbank Accession No. AAX70946), human adenovirus 52 (Genbank Accession No. ABK35065), human adenovirus 34 (Genbank Accession No. AAW33508), human adenovirus 14 (Genbank Accession No. AAW33146), human adenovirus 50 (Genbank Accession No. AAW33554), human adenovirus 2 (Genbank Accession No. AP.sub.-000196), human adenovirus 12 (Genbank Accession No. AP.sub.-000141), human adenovirus 35 (Genbank Accession No. AP.sub.-000607), human adenovirus 7 (Genbank Accession No. AP.sub.-000570), human adenovirus 1 (Genbank Accession No. AP.sub.-000533), human adenovirus 11 (Genbank Accession No. AP.sub.-000474), human adenovirus 3 (Genbank Accession No. ABB 17792), and human adenovirus type 5 (Genbank accession number D12587).

In some embodiments, the polypeptides and nucleic acid molecules of the invention have the same amino acid or nucleotide sequences as those specifically recited herein or known in the art (for example in public sequence databases, such as the Genbank database). In some embodiments the polypeptides and nucleic acid molecules of the invention may have amino acid or nucleotide sequences that are variants, derivatives, mutants, or fragments of such sequences, for example variants, derivatives, mutants, or fragments having greater than 85% sequence identity to such sequences. In some embodiments, the variants, derivatives, mutants, or fragments have about an 85% identity to the known sequence, or about an 88%, 89%, 90%, 91%, 92%, 93%, 94%, 95%, 96%, 97%, 98%, or 99% sequence identity to the known sequence. In some embodiments, a variant, derivative, mutant, or fragment of a known nucleotide sequence is used that varies in length by about 50 nucleotides, or about 45 nucleotides, or about 40 nucleotides, or about 35 nucleotides, or about 30 nucleotides, or about 28 nucleotides, 26 nucleotides, 24 nucleotides, 22 nucleotides, 20 nucleotides, 18 nucleotides, 16 nucleotides, 14 nucleotides, 12 nucleotides, 10 nucleotides, 9 nucleotides, 8 nucleotides, 7 nucleotides, 6 nucleotides, 5 nucleotides, 4 nucleotides, 3 nucleotides, 2 nucleotides, or 1 nucleotide relative to the known nucleotide sequence. In some embodiments, a variant, derivative, mutant, or fragment of a known amino sequence is used that varies in length about 50 amino acids, or about 45 amino acids, or about 40 amino acids, or about 35 amino acids, or about 30 amino acids, or about 28 amino acids, 26 amino acids, 24 amino acids, 22 amino acids, 20 amino acids, 18 amino acids, 16 amino acids, 14 amino acids, 12 amino acids, 10 amino acids, 9 amino acids, 8 amino acids, 7 amino acids, 6 amino acids, 5 amino acids, 4 amino acids, 3 amino acids, 2 amino acids, or 1 amino acid relative to the known amino acid sequence.

In those embodiments where an E4ORF1 nucleic acid or amino acid sequence is used, in some embodiments such sequences are used without other sequences from the adenovirus E4ORF1 region—for example not in the context of the nucleotide sequence of the entire E4ORF1 region or not together with other polypeptides encoded by the E4ORF1 region.

However, in some other embodiments such sequences may be used in conjunction with one or more other nucleic acid or amino acid sequences from the E4ORF1 region, such as E4ORF2, E4ORF3, E4ORF4, or E4ORF5 sequences, or variants, mutants or fragments thereof. For example, although E4ORF1 sequences can be used in constructs (such as a viral vectors) that contain other sequences, genes, or coding regions (such as promoters, marker genes, antibiotic resistance genes, and the like), in certain embodiments, the E4ORF1 sequences are used in constructs that do not contain the entire E4ORF1 region, or that do not contain other ORFs from the entire E4ORF1 region, such as E4ORF2, E4ORF3, E4ORF4, and/or E4ORF5.

The nucleic acid molecules of the invention can be used in constructs or vectors that contain various other nucleic acid sequences, genes, or coding regions, depending on the desired use, for example, promoters, enhancers, antibiotic resistance genes, reporter genes or expression tags (such as, for example nucleotides sequences encoding GFP), or any other nucleotide sequences or genes that might be desirable. The polypeptides of the invention can be expressed alone or as part of fusion proteins.

In some embodiments, nucleic acid molecules of the invention can be under the control of one or more promoters to allow for expression. Any promoter able to drive expression of the nucleic acid sequences in the desired cell type can be used. Examples of suitable promoters include, but are not limited to, the CMV, SV40, RSV, HIV-Ltr, and MML promoters. The promoter can also be a promoter from the adenovirus genome, or a variant thereof. For example, in some embodiments where E4ORF1 is used, the promoter may be a promoter that drives expression of E4ORF1 in nature in an adenovirus genome. However, in other embodiments where E4ORF1 is used, the promoter is not one that drives expression of E4ORF1 in nature in an adenovirus genome.

In some embodiments, nucleic acid molecules of the invention can be placed under the control of an inducible promoter, so that expression of the nucleic acid sequences can be turned on or off as desired. Any suitable inducible expression system can be used, such as, for example, a tetracycline inducible expression system, or a hormone inducible expression system. For example, the nucleic acid molecules of the invention can be expressed while they are needed and then switched off when the desired outcome has been achieved, for example when there has been sufficient growth or proliferation of the endothelial cells. The ability to turn on or turn off expression could be particularly useful for in vivo applications.

The nucleic acid molecules of the invention may comprise naturally occurring nucleotides, synthetic nucleotides, or a combination thereof. For example, in some embodiments the nucleic acid molecules of the invention can comprise RNA, such as synthetic modified RNA that is stable within cells and can be used to direct protein expression/production directly within cells. In other embodiments the nucleic acid molecules of the invention can comprise DNA. In embodiments where DNA is used, the DNA sequences may be operably linked to one or more suitable promoters and/or regulatory elements to allow (and/or facilitate, enhance, or regulate) expression within cells, and may be present in one or more suitable vectors or constructs. The nucleic acid molecules of the invention can be introduced into endothelial cells in the same nucleic acid construct or they can be introduced in separate nucleic acid constructs.

The nucleic acid molecules of the invention can be introduced into endothelial cells using any suitable system known in the art, including, but not limited to, transfection techniques and viral-mediated transduction techniques. Transfection methods that can be used in accordance with the present invention include, but are not limited to, liposome-mediated transfection, polybrene-mediated transfection, DEAE dextran-mediated transfection, electroporation, calcium phosphate precipitation, microinjection, and micro-particle bombardment. Viral-mediated transduction methods that can be used include, but are not limited to, lentivirus-mediated transduction, adenovirus-mediated transduction, retrovirus-mediated transduction, adeno-associated virus-mediated transduction and herpesvirus-mediated transduction.

The present invention also provides vectors, including expression vectors that contain nucleic acid molecules of the invention. For example, in one embodiment, the present invention provides an expression vector comprising a nucleotide sequence encoding BMP4 and/or an E4ORF1 polypeptide. In some such embodiments the expression vector is a viral vector. In some such embodiments the expression vector is a lentivirus vector. In some embodiments a nucleotide sequence encoding BMP4 and a nucleotide sequence encoding E4ORF1 are provided in the same construct or vector, and may be under the control of separate promoters or may be under the control of separate promoters, for example with an internal ribosome entry site sequence (IRES) between the BMP4 and E4ORF1 sequences.

In some embodiments a peptidomimetic may be used. A peptidomimetic is a small protein-like chain designed to mimic a polypeptide. Such a molecule could be designed to mimic any of the polypeptides of the invention (e.g. a BMP4 or E4ORF1 polypeptide). Various different ways of modifying a peptide to create a peptidomimetic or otherwise designing a peptidomimetic are known in the art and can be used to create a peptidomimetic of one of the polypeptides of the invention.

The handling, manipulation, and expression of the polypeptides and nucleic acid molecules of the invention may be performed using conventional techniques of molecular biology and cell biology. Such techniques are well known in the art. For example, one may refer to the teachings of Sambrook, Fritsch and Maniatis eds., “Molecular Cloning A Laboratory Manual, 2nd Ed., Cold Springs Harbor Laboratory Press, 1989); the series Methods of Enzymology (Academic Press, Inc.), or any other standard texts for guidance on suitable techniques to use in handling, manipulating, and expressing nucleotide and/or amino acid sequences. Additional aspects relevant to the handling or expression of E4ORF1 sequences are described in U.S. Pat. No. 8,465,732, the contents of which are hereby incorporated by reference.

### Endothelial Cells

The non-thymic endothelial cells (ntECs) described herein can be from any suitable non-thymic source of vascular endothelial cells known in the art. In some embodiments the ntECS do not express one or more markers (or marker profiles) that are specific to thymic endothelial cells. In some embodiments the endothelial cells are primary endothelial cells. In some embodiments the endothelial cells are mammalian cells, such as human or non-human primate cells, or rabbit, rat, mouse, goat, pig, or other mammalian cells. In some embodiments the endothelial cells are primary human endothelial cells. In some embodiments the endothelial cells are umbilical vein endothelial cells (UVECs), such as human umbilical vein endothelial cells (HUVECs). In some embodiments the endothelial cells are adipose ECs. In some embodiments the endothelial cells are skin ECs. In some embodiments the endothelial cells are cardiac ECs. In some embodiments the endothelial cells are kidney ECs. In some embodiments the endothelial cells are lung ECs. In some embodiments the endothelial cells are liver ECs. In some embodiments the endothelial cells are bone marrow ECs. Other suitable endothelial cells that can be used include those described previously as being suitable for E4ORF1-expression in U.S. Pat. No. 8,465,732, the contents of which are hereby incorporated by reference.

In some embodiments the endothelial cells are gene-modified such that they comprise one or more genetic modifications in addition to and apart from the expression of the specific recited molecules (e.g. BMP4 and/or E4ORF1). For example, in some embodiments the endothelial cells may also be engineered to express ETV2. Indeed, in each of the embodiments described throughout this patent disclosure where the ntECs express BMP4 and/or E4ORF1, the ntECs may also express ETV2. Furthermore, in some embodiments the ntECs described herein may comprise a corrected version of a gene known to be involved in, or suspected of being involved in, a disease or disorder that affects endothelial cells, or any other gene, such as a therapeutically useful gene, that it may be desired to provide in endothelial cells or administer or deliver using engineered endothelial cells.

### Cell Populations & Compositions

The endothelial cells of the invention may exist in, or be provided in the form of, a population of endothelial cells or a composition comprising such a population of endothelial cells.

For example, in one embodiment, the present invention provides a population of engineered BMP4+E4ORF1+ non-thymic endothelial cells (ntECs). In some embodiments such populations of engineered ntECs are in vitro or ex vivo. In some embodiments such populations of engineered ntECs are in vivo. In some embodiments such populations of engineered ntECs are isolated populations. In some embodiments such populations are substantially pure populations of ntECs cells. For example, in some embodiments at least about 50%, preferably at least about 75%, more preferably at least about 85%, and most preferably at least about 95% of the cells making up a total cell population will be the engineered ntECs of the invention. In some embodiments the ntECs in the population of engineered ntECs are umbilical vein endothelial cells (UVECs), adipose endothelial cells, skin endothelial cells, lung endothelial cells, heart endothelial cells, kidney endothelial cells or bone marrow endothelial cells. In some embodiments the ntECs are human umbilical vein endothelial cells (HUVECs).

In some embodiments the present invention provides various compositions comprising the populations of ntECs described above, or elsewhere herein. In some embodiments such compositions comprise a carrier solution, such as a physiological saline solution, cell suspension medium, cell culture medium, or the like. In some embodiments such compositions are therapeutic compositions comprising a population of ntECs as described herein and a solution suitable for administration to a subject, such as a physiological saline solution. Other therapeutically acceptable agents can be included if desired. One of ordinary skill in the art can readily select suitable agents to be included in the therapeutic compositions depending on the intended use. In some embodiments such compositions and therapeutic compositions may comprise a population of ntECs as described herein and a biocompatible matrix material (such as a biocompatible matrix material that is liquid or solid at room temperature or at body temperature).

In some embodiments the compositions described herein may comprise additional cell types—such as, for example, additional cell types that can be maintained, cultured, or expanded in the presence of the ntECs (e.g. using the ntECs as “feeder” cells), or administered to a subject together with the ntECs. Examples of such additional cell types include, but are not limited to, stem or progenitor cells, such as thymic stem or progenitor cells, hematopoietic cells, hematopoietic stem cells (HSCs), hematopoietic progenitor cells (HPCs) and hematopoietic stem and progenitor cells (HSPCs). Other examples of cells that can be provided or used together with the endothelial cells of the invention are provided in U.S. Pat. No. 8,465,732, the contents of which are hereby incorporated by reference.

### Methods of Treatment and Other Applications

In some embodiments, the present invention provides various therapeutic methods, such as methods for treating subjects in need thereof by administering to such subjects an effective amount of the engineered ntECs of the invention (or a composition comprising such engineered ntECs).

For example, in one embodiment the present invention provides a method of enhancing thymic regeneration, the method comprising administering an effective amount of a therapeutic composition comprising engineered non-thymic endothelial cells (ntECs) to a subject in need of thymic regeneration, wherein the engineered ntECs are either E4ORF1+ or BMP4+E4ORF1+, thereby enhancing thymic regeneration in the subject.

In another embodiment the present invention provides a method of increasing survival following myeloablative conditioning treatment in a subject, the method comprising administering an effective amount of a therapeutic composition comprising engineered non-thymic endothelial cells (ntECs) to a subject who has undergone myeloablative conditioning, wherein the engineered ntECs are BMP4+E4ORF1+. Similarly, in another embodiment the present invention provides a method of increasing survival following myeloablative conditioning treatment and subsequent hematopoietic cell transplant (HCT) (e.g. hematopoietic stem cell transplant (HSCT)) in a subject, the method comprising administering an effective amount of a therapeutic composition comprising engineered non-thymic endothelial cells (ntECs) to a subject who has undergone myeloablative conditioning and a subsequent hematopoietic cell transplant (HCT) (e.g. hematopoietic stem cell transplant (HSCT)), wherein the engineered ntECs are BMP4+E4ORF1+. In such methods, survival is increased as compared to that in subjects that undergo the same myeloablative conditioning treatment (and/or HCT) but that do not receive the engineered ntECs.

Means for selecting an appropriate effective amount of ntECS, or a composition comprising ntECs, are described above.

The engineered non-thymic endothelial cells (ntECs) can be administered to the subject once (a single administration) or multiple times (multiple administrations), for example, 2, 3, or 4 administrations. Where multiple administrations are employed, the schedule of administrations may be any suitable schedule. In some embodiments the multiple administrations are 1 day apart, 2 days apart, 3 days apart, 3 days apart, or 5 days apart. For example, in one embodiment, the ntECs are administered on day 0, day 3 and day 5. The appropriate time for first administering the ntECs to the subject (i.e. day 0) can be selected by a physician based on the subject's particular circumstances. For example, in one embodiment, if the subject has been treated with a myeloablative conditioning treatment in preparation for receiving a hematopoietic cell transplant (HCT), the ntECs are first administered to the subject on the same day that the subject receives the HCT (day 0), and ntECs may or may not be administered to the subject again on subsequent days, for example on days 3 and 5 post HCT. In some embodiments the effective amount of the ntECs is administered to the subject once—in a single administration. In some embodiments the effective amount of the ntECs is administered to the subject multiple times—i.e. multiple administrations, each of an effective amount of ntECs, are delivered to the subject. In some embodiments the effective amount of the ntECs is split amongst multiple administrations—for example, half of the effective amount is administered on one day in a first administration and half of the effective amount is administered on another day in a second administration. Numerous variations of these administration schemes can be employed, as appropriate. The skilled artisan will be able to select a suitable administration schedule depending on the particular situation.

In some embodiments thymic regeneration comprises recovery of at least one cell type from among CD45− thymic stromal cells and CD45+ T cells. CD45− thymic stromal cells include, but are not limited to, thymic epithelial progenitors (TEPCs), cortical thymic epithelial cells (cTECs), and medullary thymic epithelial cells (mTECs). CD45+ T cells include, but are not limited to, CD3+ T cells, CD4+ T cells, CD8+ T cells, double-positive T cells (DP), double-negative T cells (DN), double-negative type 1 (DN1) T cells, double-negative type 2 (DN2) T cells, double-negative type 3 (DN3) T cells and double-negative type 4 (DN4) T cells. In some embodiments the thymic regeneration comprises recovery of both CD45− thymic stromal cells and CD45+ T cells. In some embodiments the thymic regeneration comprises recovery of thymic epithelial progenitors (TEPCs), cortical thymic epithelial cells (cTECs), and medullary thymic epithelial cells (mTECs). In some embodiments the thymic regeneration comprises recovery of CD4+ T cells, CD8+ T cells, double-positive T cells (DP), double-negative T cells (DN), Double-negative type 1 (DN1) T cells, Double-negative type 2 (DN2) T cells, and Double-negative type 4 (DN4) T cells. In some embodiments the thymic regeneration comprises recovery of thymic epithelial progenitors (TEPCs), cortical thymic epithelial cells (cTECs), and medullary thymic epithelial cells (mTECs), CD4+ T cells, CD8+ T cells, double-positive T cells (DP), double-negative T cells (DN), Double-negative type 1 (DN1) T cells, Double-negative type 2 (DN2) T cells, and Double-negative type 4 (DN4) T cells.

In some embodiments the ntECs are selected from the group consisting of umbilical vein endothelial cells (UVECs), adipose endothelial cells, skin endothelial cells, lung endothelial cells, heart endothelial cells, kidney endothelial cells and bone marrow endothelial cells.

In some of embodiments the subjects are human. In some of such embodiments the ntECs are human umbilical vein endothelial cells (HUVECs). In some of such embodiments the ntECs are human bone marrow endothelial cells. In some of such embodiments the ntECs are human adipose endothelial cells. In some of such embodiments the ntECs are human skin endothelial cells.

In some embodiments the ntECs are autologous to the subject. In some embodiments the ntECs are allogeneic to the subject. In some embodiments the ntECs are MHC/HLA-matched to the subject.

In some embodiments the subjects have previously been treated with chemotherapy, radiation therapy, a pre-transplantation conditioning regimen, or a myeloablative conditioning regimen. Examples of myeloablative conditioning regimens include, but are not limited to, those involving treating subjects with radiation (e.g. total body irradiation) and/or administering etoposide and/or busulfan to the subject.

In some embodiments the subjects have previously been treated with a hematopoietic cell transplantation (HCT), a hematopoietic stem cell transplant (HSCT) or a hematopoietic stem and/or progenitor cell transplant (HSPCT).

In some embodiments the subject has an immunodeficiency. In some embodiments the subject has an HIV infection. In some embodiments the subject has an ageing-related deficiency in thymic tissue mass, thymic function, or T-cell production.

In some embodiments the methods further comprise administering to the subject a therapeutic composition comprising hematopoietic cells or hematopoietic stem cells (HSCs) or hematopoietic stem and progenitor cells (HSPCs), e.g. in the context of performing a hematopoietic cell transplant (HCT) (e.g. a hematopoietic stem cell transplant (HSCT) or a hematopoietic stem and/or progenitor cell transplant (HSPCT)) procedure. In some of such embodiments the engineered ntECs are administered to the subject by IV infusion. In some of such embodiments the engineered ntECs and the hematopoietic cells (e.g. HSCs) are administered concurrently. In some of such embodiments the engineered ntECs and the hematopoietic (e.g. HSCs) are administered to the subject in the same infusion. In some of such embodiments the engineered ntECs are administered to the subject in multiple IV infusions over the course of several days or weeks.

In some embodiments of the treatment methods provided herein, BMP4 protein is administered to the subjects. For example, in some such embodiments BMP4 protein is added to a composition containing E4ORF1+ or BMP4+E4ORF1+ ntECs prior to administering such a composition to a subject. In some embodiments a separate composition comprising BMP4 protein is administered to the subject as part of the treatment method. In some embodiments of the treatment methods provided herein, BMP4 is not administered to the subjects.

In some embodiments thymic endothelial cells are also administered to the subjects (i.e. both non-thymic ECs (ntECs) and thymic ECs are administered). In some embodiments thymic endothelial cells are not administered to the subjects.

In the treatment methods provided herein, the ntECs or ntEC-containing compositions can be administered to subjects using any suitable means known in the art, for example by injection (e.g. intravenous (IV) injection, intramuscular injection, subcutaneous injection, local injection), by infusion (e.g. by IV infusion, subcutaneous infusion, local infusion), or by surgical implantation. In some embodiments the ntECs or ntEC-containing compositions are administered to subjects by IV infusion. For example, in some embodiments the engineered ntECs of the present invention may be administered directly into, or in the vicinity of, the thymus. In some embodiments the engineered ntECs of the present invention may be administered to subjects by intra-thymic injection or intra-thymic infusion. In some embodiments the engineered ntECs of the present invention may be administered to subjects by injection or infusion into the inferior thyroid artery. In some embodiments the engineered ntECs of the present invention may be administered to subjects by injection or infusion into the internal thoracic artery. The skilled artisan will be able to select a suitable route of administration depending on the particular situation.

In some embodiments the thymus can be made more permissive to homing of the engineered ntECs to the thymus via mechanical, magnetic, ultrasound, or other stimulatory methods.

In some embodiments the engineered ntECs of the present invention can be created in vivo, for example for research purposes or for therapeutic applications. For example, in some aspects, the present invention provides various therapeutic methods, such as methods for treating subjects in need thereof, which comprise administering to such subjects an effective amount of a nucleic acid molecule that encodes BMP4 and/or a nucleic acid molecule that encodes E4ORF1 (for example in a suitable vector, and/or under the control of a suitable promoter) such that non-thymic endothelial cells in the subject are transfected or transduced with such nucleic acid molecules and become engineered ntECs in vivo. In such methods, the nucleotide molecules can be administered to subjects using any suitable means known in the art. For example, the nucleotide molecules (for example in a suitable vector) can be administered by injection or infusion into the blood stream or tissue at a desired location. The nucleic acid molecules can be administered in a single dose or in multiple doses. The skilled artisan will be able to select a suitable method of administration according and a suitable dosing regimen depending on the desired use.

In some embodiments the engineered ntECs of the invention are mitotically inactivated prior to use (e.g. therapeutic use) such that they cannot replicate. This can be achieved, for example, by using a chemical agent such as mitomycin C or by irradiating the engineered endothelial cells.

In some embodiments the treatment methods of the present invention further comprise an initial step of genetically modifying ntECs by transducing or transfecting the ntECs in vitro or ex vivo with a nucleic acid molecule encoding E4ORF1 and optionally a nucleic acid molecule encoding BMP4, prior to administering the ntECs to the subject.

Any of the various treatments methods described herein can be used to enhance thymic regeneration (including stimulating T-cell reconstitution) and/or achieve any one or more of the parameters or treatment outcomes listed in the “treatment” definition section above—in a living subject in need thereof.

Cell Culture Methods

Methods of culturing cells are well known in the art and any suitable cell culture methods can be used. For example, the engineered ntECs of the invention can be cultured using methods known to be useful for culturing other endothelial cells, or, methods known to be useful for culturing E4ORF1-expressing endothelial cells, for example as described in U.S. Pat. No. 8,465,732, the contents of which are hereby incorporated by reference. In some embodiments the engineered endothelial cells of the invention can be cultured in the absence of serum, or in the absence of exogenous growth factors, or in the absence of both serum and exogenous growth factors. The engineered endothelial cells of the invention can also be cryopreserved. Various methods for cell culture and cell cryopreservation are known to those skilled in the art, such as the methods described in Culture of Animal Cells: A Manual of Basic Technique, 4th Edition (2000) by R. Ian Freshney (“Freshney”), the contents of which are hereby incorporated by reference.

### Kits

The present invention also provides kits for carrying out the various methods described herein or for producing the engineered endothelial cells provided herein. Such kits may contain any of the components described herein, including, but not limited to, nucleotide sequences (for example in a vector), ntECs, populations of engineered ntECS, control non-engineered ntECs, sample/standard engineered ntECs, means or compositions for detection of engineered ntECs or the proteins or nucleic acid molecules expressed therein, (e.g. nucleic acid probes, antibodies, etc.), media or compositions useful for maintaining or expanding engineered ntECs, media conditioned by engineered ntECs, means or compositions for administering engineered endothelial cells to a subject, or any combination thereof. All such kits may optionally comprise instructions for use, containers, culture vessels and the like. A label may accompany the kit and may include any writing or recorded material, which may be electronic or computer readable form (e.g., disk, optical disc, memory chip, or tape) providing instructions or other information for use of the kit contents.

Certain aspects of the present invention may be further described in the following non-limiting Examples.

### EXAMPLES

### Example 1

### Generation of Engineered Non-Thymic Endothelial Cells

### Isolation of Non-Thymic Endothelial Cells

Non-thymic endothelial cells (ntECs) are isolated from a desired tissue, such as from umbilical cord/umbilical veins, adipose tissue, skin, lung, heart, kidney or bone marrow, or any other desired non-thymic tissue source. Endothelial cells are isolated from the desired tissue using standard established protocols. An exemplary protocol for isolation of ntECS from umbilical cord is provided below. Similar protocols are known for the isolation of endothelial cells from other tissue sources and can be used.

The following is an exemplary protocol for the isolation of ntECs from umbilical cord veins from any desired species, such as humans. This protocol generates populations of umbilical vein endothelial cells or “UVECs”—which, if derived from human umbilical vein, are referred to as “HUVECs.” All steps are performed using aseptic technique and aseptic materials.

Umbilical cords are maintained at 2-8° C. until they are processed for isolation of endothelial cells. UVECs are isolated from the umbilical vein of the umbilical cords by enzymatic digestion with collagenase. For example, the umbilical vein can be flushed with a physiological saline or other solution suitable for living cells (e.g. culture medium) and then filled with a solution of 0.2% (w/v) collagenase in a physiological saline or other solution suitable for living cells (e.g. culture medium) and clamped at both ends. The cord is incubated at a suitable temperature (e.g. room temperature 20-25° C.) for sufficient time to allow the dissociation of the endothelial cells from the umbilical vein (e.g. about or 30 minutes at room temperature). The amount of time may be varied depending on the temperature at which the incubation occurs and based on the particular properties of the tissue. The detached cells are flushed out of the vein and retained in a suitable container such as a conical tube. The umbilical vein may be washed again with a physiological saline or other solution suitable for living cells (e.g. culture medium) and the wash volume also retained—for example in the same container. The contents of the container are centrifuged, and the supernatant is aspirated. The cell pellet comprising UVECs is gently resuspended in an EC culture medium. An exemplary culture medium that can be used is an EC growth medium comprising M199 base medium supplemented with 10% fetal bovine serum, 20 ng/mL FGF-2, 10 U/mL heparin, 5 μg/mL Gentamicin, 10 mM HEPES and 1× Glutamax. The UVECs in EC culture medium are transferred to a suitable tissue culture vessel and placed in an incubator at 37° C., 5% CO2 to begin the in vitro culture process. One of skill in the art will recognize that variations can be made to this protocol to achieve isolation of UVECs. One of skill in the art will also recognize that variations of this protocol can be made to isolate ntECs from other tissue sources.

Transfection or Transduction to Produce Engineered ntECs

After a suitable period of time in culture (e.g. 2 days) ntECs isolated as described above, or isolated or obtained by any other means, are transfected or transduced with a nucleic acid molecule (e.g. in an expression vector, viral vector, etc.) containing a selectable marker and the desired coding sequence(s) (e.g. an E4ORF1 coding sequence (e.g. from Ad5) or a BMP4 coding sequence) under the control of a suitable promoter. The transfection or transduction is performed using standard transfection or transduction protocols. If a second coding sequence is to be transfected or transduced (e.g. an E4ORF1 coding sequence (e.g. from Ad5) or a BMP4 coding sequence), after a suitable period of time (e.g. the next day) the ntECs are transfected or transduced with a second nucleic acid molecule (e.g. in an expression vector, viral vector, etc.) containing a selectable marker and the second coding sequence under the control of a suitable promoter—again using standard transfection or transduction protocols. When there are two transfections/transductions the order of the transfections/transductions can be as desired (e.g. E4ORF1 first or second). The two coding sequences can also be delivered at the same time—whether in the same nucleic acid molecule or in separate nucleic acid molecules.

Typically, after transfection or transduction the ntECs cells are maintained in EC culture medium for several days (e.g. 3 days) before switching to a suitable selection medium containing an appropriate selection agent or agents. For example, if the delivered nucleic acid molecule comprises a gene that confers resistance to a given antibiotic, a suitable amount of that antibiotic is provided in the selection medium—such that cells that do not comprise the nucleic acid molecule die and cells that do comprise the nucleic acid molecule survive.

Typically, the ntECs are also subjected to serum starvation culture (E4ORF1 expression confers on the ntECs an ability to survive in serum free culture) for a certain period of time. The ntECs are then expanded in a suitable EC culture medium using standard EC culture protocols for sufficient time to produce a sufficient number of cells for the desired use. For example, in the case of UVECs an expansion phase of 14-21 days can readily yield around 200-300×10⁶ E4ORF1+ UVECs from one umbilical cord. The amount of starting material and the expansion phase can be adjusted as need be to yield the desired amount of ntECs. If desired bioreactor systems can be used to produce large quantities of ntECs under controlled conditions. For example, a Quantum Cell Expansion System (Quantum, Terumo BCT) bioreactor system can be used. One of skill in the art will recognize that variations can be made to the protocols described above to generate a suitable population of engineered ntECs for the desired use (e.g. E4ORF1+, BMP4+ or BMP4+E4ORF1+ ntECs).

Engineered ntEC Cryopreservation

While engineered ntECs generated as above, or generated using other means, can be used immediately, or maintained in culture until they are to be used, in some situations it may be desirable to cryopreserve the ntECS and/or to generate an ntEC cell bank—for example so that the ntECs can be used at a later time. Various cryopreservation protocols and cryopreservation reagents that are suitable for use with endothelial cells are known in the art, and any such methods/reagents can be used. For example, engineered ntECs can be cryopreserved using CryoStor CS5 cryopreservation reagent (BioLife Solutions, Seattle, Wash.), optionally supplemented with Human Serum Albumin (HSA: from Griffols) to a final concentration of about 20% HSA. The cryopreserved ntECs can be stored frozen until needed. Typically, when needed, the ntECs will be thawed, transferred to an EC culture medium, and expanded in culture to the desired degree.

Engineered ntEC Quality Control

Quality control tests can be performed on the engineered ntECS at any step during the production process and/or prior to use—if desired. For example, quality control assays can be performed to evaluate and confirm cell viability, lack of contamination, presence of BMP4 expression (e.g. by RT PCR), presence of BMP4 secretion (e.g. by ELISA), presence of E4ORF1 expression (e.g. by RT PCR), and the like. For example, an exemplary quality control data assessment was performed based on detection of secreted BMP4 protein as detected by ELISA. Conditioned media was harvested from wells containing BMP4+ E4ORF1+ HUVECs. BMP4 was detectable in the conditioned media at a mean concentration of approximately 60,000 (n=2 independent lots, run in duplicate). In another quality control assessment performed on four different BMP4+ E4ORF1+ HUVECs lines the mean concentration of BMP4 in the conditioned medium was approximately 29,104 pg/mL for the first line, approximately 4,542 pg/mL for the second line, approximately 1,798 pg/mL for the third line, and approximately 3,211 pg/mL for the fourth line.

### Example 2

### Resistance of Non-Thymic Endothelial Cells to Serum Starvation

Human umbilical vein endothelial cells were isolated and placed in culture in complete medium (medium supplemented with 20% fetal calf serum and fibroblast growth factor-2; FGF-2) until they were confluent. Cells were then split into six different wells each containing 100,000 endothelial cells with complete medium. Cells in Wells 1 and 2 were used as controls (no transfection); Cells in Wells 3 and 4 were transfected with a retrovirus carrying the BMP4 gene. Cells in wells 5 and 6 were transfected with a retrovirus carrying the E4ORF1 region of an adenovirus—which is known to confer resistance to serum-starvation.

All cells in wells 2, 4, and 6 were then switched to serum-free medium for 4 days; wells 1, 3, and 5 were maintained in complete medium for four days. At the end of 4 days each well was examined to determine cell growth. The results are summarized in Table 1, below.

TABLE 1

| Well | Transduction | Serum Starvation | Outcome |
|---|---|---|---|
| 1 | No transduction | Normal serum | Positive growth |
| 2 | | Serum starvation | No growth |
| 3 | Transduction with EBMP4 | Normal serum | Positive growth |
| 4 | | Serum starvation | No growth |
| 5 | Transduction with E4ORF1 | Normal serum | Positive growth |
| 6 | | Serum starvation | Positive growth |

The results showed that serum starvation resulted in loss of cell growth in the absence of gene transduction (wells 1 and 2). Transduction with BMP4 did not confer resistance to serum starvation (wells 3 and 4), but transduction with adenoviral E4ORF1 did (wells 5 and 6).

The results of these studies are further summarized in Table 2, below, which shows (in a relative form) the effect of transfection of HUVECs with E4ORF1 alone, BMP4 alone, or both E4ORF1 and BMP4, on the ability of HUVECs to thrive in the absence of serum. The “−” and “+” symbols provide an approximate representation of the cell numbers, with more “+” symbols representing greater cell numbers.

TABLE 2

| | | | | HUVECs |
|---|---|---|---|---|
| In vitro | | HUVECs | HUVECs | E4ORF1+ |
| outcome | HUVECs | E4ORF1+ | BMP4+ | BMP4+ |
| Ability to thrive | − | ++++ | − | ++++ |
| in absence of | (cells died) | | (cells died) | |
| serum | | | | |

### Example 3

### Growth of Non-Thymic Endothelial Cells in the Presence of Serum

In this study human umbilical vein endothelial cells were isolated and placed in culture in complete medium (medium supplemented with 20% fetal calf serum and fibroblast growth factor-2; FGF-2) until they were confluent. Cells were then split into four different well each containing 100,000 endothelial cells with complete medium. Cells in Well 1 were used as a control (no transfection); Cells in Well 2 transfected with a retrovirus carrying the BMP4 gene, Cells in wells 3 were transfected with a retrovirus carrying the E4ORF1 region of Adenovirus (which is known to confer a growth advantage to the cells).

All cells were cultured to confluence then passaged, re-plated at 150,000 cells per well, and cultured for 15 days. All cultures were passaged every 4-6 days. Cell number was determined at each passage using trypan blue dye exclusion and a hemocytometer.

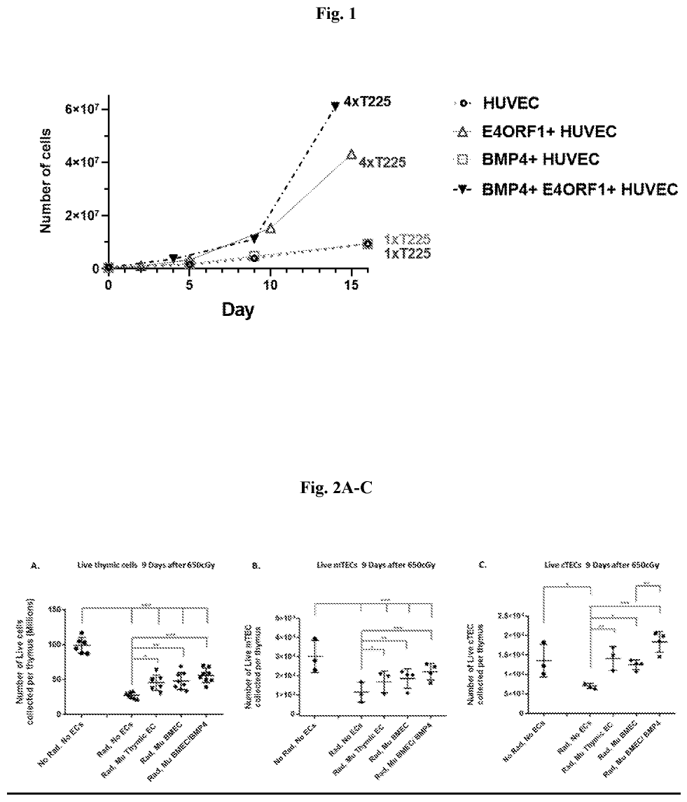

The results showed that cells transduced with BMP4 alone grew no faster than those that were not transduced whereas cells transduced with E4ORF1 exhibited significantly faster growth rate. See FIG. 1. Thus, transduction of human umbilical vein endothelial cells with BMP4 does not confer a substantial increase in growth rate to the cells. By contrast, transduction with adenovirus E4ORF1 region confers a substantial growth advantage. The results of these studies are further summarized in Table 3, below, which shows (in a relative quantitative form) the effect of transfection with E4ORF1 alone, BMP4 alone, or both E4ORF1 and BMP4, on the proliferative capacity of HUVECs grown in the presence of serum. The “+” symbols provide an approximate representation of the cell numbers, with more “+” symbols representing greater cell numbers. For the actual cell numbers please refer to the corresponding data figures.

TABLE 3

| | | | | HUVECs |
|---|---|---|---|---|
| In vitro | | HUVECs | HUVECs | E4ORF1+ |
| outcome | HUVECs | E4ORF1+ | BMP4+ | BMP4+ |
| Proliferative | + | ++++ | + | ++++ |
| capacity in the | (limited | (robust | (limited | (robust |
| presence of | proliferation) | proliferation) | proliferation) | proliferation) |
| serum | | | | |

### Example 4

### BMP4+E4ORF1+ Non-Thymic Endothelial Cells Stimulate Thymic Epithelial Regeneration In Vivo

BMP4+E4ORF1+ ntECs were generated essentially as described in the previous Examples. C57Bl/6 mice were exposed to 650 Rads of total body irradiation (TBI), a sub-lethal dose that is sufficient to induce systemic hematopoietic and thymic damage. Mice were injected with 1×10⁶ mouse thymic endothelial cells (mutECs) or with BMP4+E4ORF1+ bone marrow endothelial cells (referred to as “muBMECs+BMP4” in FIG. 2) suspended in a physiological saline (phosphate buffered saline—“PBS”) 6 hours post-irradiation. There was also a “no EC” infusion control group of mice injected with PBS alone. Tissues were harvested 9 days post TBI. The number of live thymic cells, live medullar thymic epithelial cells (mTEC), and recovered live cortical thymic epithelial cells (cTEC) was determined. The results are presented in FIG. 2 in which the number of live thymic cells (FIG. 2A), live medullar epithelial cells (mTEC) (FIG. 2B), and live cortical epithelial cells (cTEC) (FIG. 2C) recovered is plotted on each graph. The data shows that mouse BMP4+E4ORF1+ bone marrow endothelial cells accelerate thymic recovery after sub-lethal irradiation.

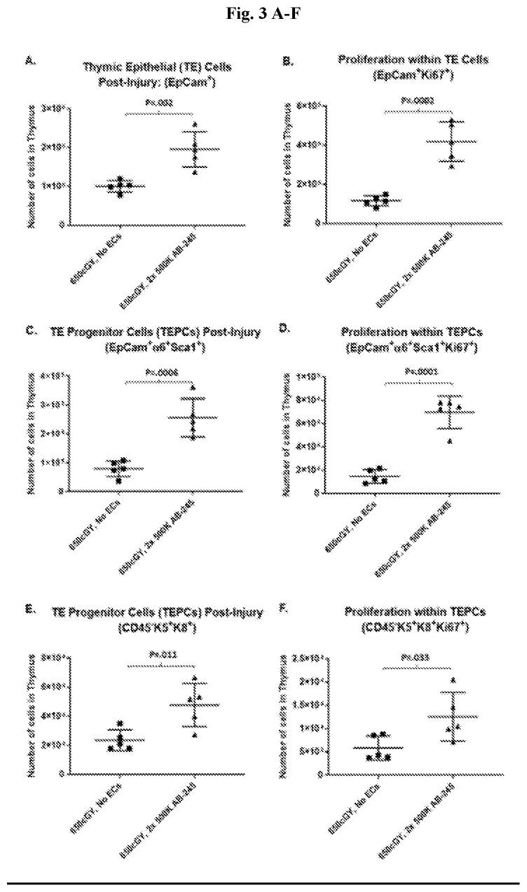

In order to discern the role of different subsets of thymic epithelial cells (TECs)—i.e. mTECs and cTECs, in thymic regeneration, additional studies were performed to identify the cell types affected by the BMP4+ E4ORF1+ ntEC treatment (referred to in FIG. 3 as “AB245” cells). C57Bl/6 mice were exposed to 650 Rads of TBI to induce thymic damage. Control irradiated mice were compared to those that also received BMP4+E4ORF1+ HUVECs at days 1 and 3 post-irradiation. Animals were sacrificed at day 4 post irradiation to observe the effects of the BMP4+E4ORF1+ HUVEC transplants. At this early time point, transplanted animals exhibited nearly double the amount of overall thymic epithelial content (measured by number of CD45− EpCam+ cells) as compared to non-transplanted animals. (FIG. 3A), with a near four-fold increase in the total number of these cells actively proliferating (CD45− EpCam+ Ki67+) (FIG. 3B). The number of thymic epithelial progenitor cells (TEPCs) identified by markers EpCam+, alpha6 integrin+, and Sca1+ (FIG. 3C) and CD45−, K5+, K8+ cells (FIG. 3E) was significantly increased compared to animals that received no cellular transplant. Proliferating populations of these TEPCs also increased and were found to be 2-fold to 4-fold greater in number in animals that received BMP4+ E4ORF1+ HUVECs as compared to animals that received no cellular transplant (FIG. 3, D, F).

The results of the above studies are further summarized in Tables 4A and 4B, below, which show (in a relative form) the ability of the indicated ntEC types to accelerate recovery of the listed thymic cell type(s) in the thymus following sublethal irradiation (see “treatment outcome” column). The data summarized in Table 4A (data from FIG. 2) summarizes recovery induced by administration of BMP4+E4ORF1+ bone marrow ECs. The data summarized in Table 4B (data from FIG. 3) summarizes recovery induced by administration of BMP4+E4ORF1+ HUVECs. The “+” symbols provide an approximate representation of the cell numbers, with more “+” symbols representing greater cell numbers. For the actual cell numbers please refer to the corresponding data figures.

TABLE 4A

| Treatment Outcome | | | | |
|---|---|---|---|---|
| (number of thymic cells | | Mouse | BMP4+E4ORF1+ | |
| following recovery from | No | Thymic | Mouse Bone | |
| irradiation, by cell type) | ECs | ECs | Marrow ECs | Data FIG. |
| Recellularization (total | + | ++ | ++++ | FIG. 2A |
| thymic cells) | | | | |
| mTEC | + | ++ | ++++ | FIG. 2B |
| cTEC | + | ++ | ++++ | FIG. 2C |

TABLE 4B

| Treatment Outcome | | | | | |
|---|---|---|---|---|---|
| (number of thymic | | | | | |
| cells following | | | | | |
| recovery from | No | | BMP4+ | BMP4+E4ORF1+ | Data |
| irradiation, by cell type) | ECs | HUVEC | HUVEC | HUVEC | FIG. |
| EpCAM⁺ | + | * | * | ++++ | FIG. 3A |
| EpCAM⁺ Ki67+ | + | * | * | ++++ | FIG. 3B |
| EpCAM⁺ α6+Sca1+ | + | * | * | ++++ | FIG. 3C |
| EpCAM⁺ α6+Sca1+ | + | * | * | ++++ | FIG. 3D |
| Ki67+ (proliferating) | | | | | |
| CD45−K5+K8+ | + | * | * | ++++ | FIG. 3E |
| CD45−K5+K8+ Ki67+ | + | * | * | ++++ | FIG. 3F |
| (proliferating) | | | | | |

* HUVECs and BMP4+ HUVECs (without E4ORF1) could not be grown in sufficient number to use in this in vivo study (see FIG. 1)

### Example 5

### Engineered Non-Thymic Endothelial Cells Stimulate Recovery of Both Thymic T Cells and Thymic Epithelial Cells

The ability of engineered ntECs to stimulate lymphoid reconstitution was studied using a mouse (C57Bl/6 mice) hematopoietic stem cell transplant model. Engineered ntECs (either E4ORF1+, BMP4+, or BMP4+E4ORF1+ HUVECs) were generated essentially as described in previous Examples. Animals were assigned to treatment and control groups, as follows:

Group 1 (control)—Sixteen mice were designated as controls; these animals did not receive irradiation, infusion of bone marrow, or infusion of endothelial cells.

Groups 2-4 (treatments)—Mice were irradiated with 1,000 cGy total body irradiation. Approximately 10-16 hours after irradiation all irradiated mice received intravenous infusion of 500,000 Bone Marrow (BM) cells. The treatment groups were as follows:

Group 2—Animals received infusion of 500,000 syngeneic bone marrow cells only.

Group 3—Animals received infusion of 500,000 syngeneic bone marrow cells and 500,000 human umbilical vein endothelial cells engineered to express the E4ORF1 region of adenovirus. An additional intravenous infusion of 500,000 of these endothelial cells was infused approximately 48 hours after the first infusion and another approximately 48 hours after the second infusion.

Group 4—Animals underwent the same procedures as for Group 3 except that the endothelial cells they received at each infusion were transduced with both E4ORF1 and BMP4.

Three weeks after irradiation all animals were euthanized. The thymus of each animal was excised, dissected into small fragments and subjected to collagenase digestion to release a single cell suspension comprising the cellular components of the thymus. These cells were then subjected to flow cytometric analysis to characterize the major hematopoietic and epithelial cell types present. The results were as follows.

### A. Recellularization—Total Cell Recovery

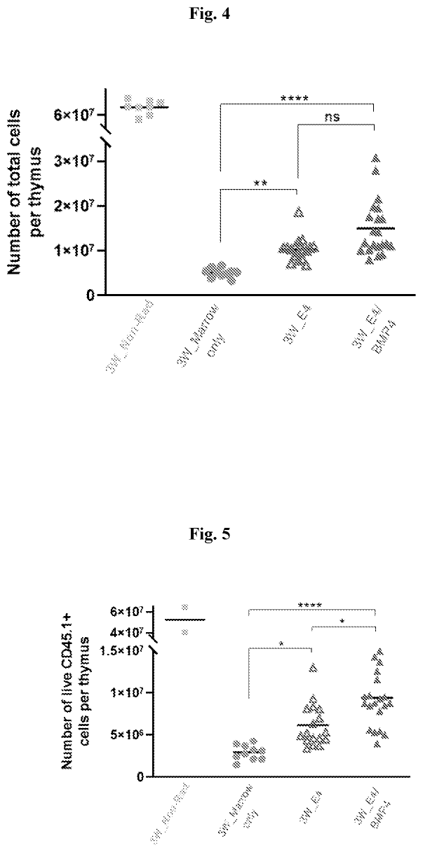

The number of nucleated thymic cells was reduced by more than 90% in irradiated animals receiving bone marrow infusion alone (FIG. 4). While still significantly lower than non-irradiated animals, total cellularity was significantly improved compared to bone marrow alone by co-infusion of ntECs transduced with E4ORF1 alone or ntECs transduced with E4ORF1 and BMP4 (FIG. 4; p<0.05 for the difference with ntECs transduced with E4ORF1 alone and p<0.01 for the difference with ntECs transduced with both E4ORF1 and BMP4).

Total cellularity in animals receiving ntECs transduced with the combination of E4ORF1 and BMP4 was numerically greater than that with those receiving ntECs transduced with E4ORF1 alone—though this difference was not statistically significant.

Data from groups treated with BM-only, E4ORF1+ ntECs, or E4ORF1+BMP4+ ntECs of all cell lots were pooled together and were analyzed with a One-way Kruskal-Wallis ANOVA and followed up with Dunn's Multiple comparison analysis test. For the Dunn's test the mean value of each group were compared with the mean of the control bone marrow—only treated group. The data is shown in FIG. 4.

### B. Recovery of Cells of Hematopoietic Origin and Thymic Stromal Cells

Cells in the thymus, a primary lymphoid organ important for T-lymphocyte development, may be divided into the cells of hematopoietic origin (those derived from CD45⁺ bone marrow hematopoietic stem cells) and thymic stromal (epithelial) cells. Recovery of these two populations, and subpopulations of each, was assessed separately—as summarized below.

### C. CD45⁺ Cell Recovery

Use of donor bone marrow derived from C57Bl/6 mice that express the CD45.1 isoform and host mice that express the CD45.2 isoform allowed discrimination between donor and host-derived CD45⁺ cells.

The number of donor CD45+ cells in the thymus 3 weeks after irradiation (FIG. 5) generally reflected total cell numbers (FIG. 4). As with total cell number the number of CD45+ cells in the thymus of ntEC-treated animals was significantly greater than that in animals receiving bone marrow infusion alone. Infusion of ntECs transduced with both E4ORF1 and BMP4 led to a greater degree of CD45+ cell recovery that seen with ntECs transduced with E4ORF1 alone. This difference was statistically significant (FIG. 5).

### D. CD3⁺ Cell Recovery

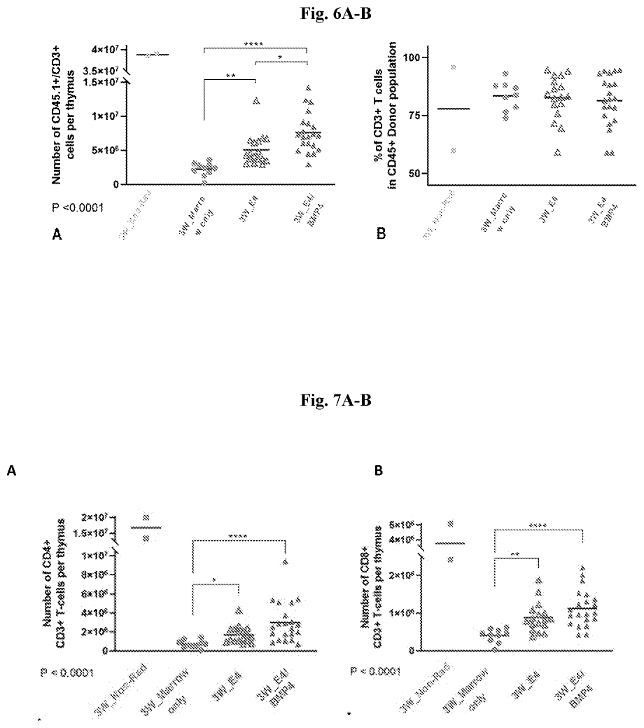

T-lymphocytes are characterized by expression of the CD3 surface marker. Recovery of the CD45.1⁺/CD3⁺ T cell population followed the same pattern as that of CD45 recovery (FIG. 6). The association with CD45 recovery is not surprising given that the majority of CD45+ cells in the thymus are T-cells (FIG. 6B). As with CD45+ cells, the greatest number of T-cells was seen in the group treated with ntECs transduced with both E4ORF1 and BMP4. The number of T-cells in this group was statistically significantly greater than that of the group treated with ntECs transduced with E4ORF1 alone.

Importantly, no between-group differences were noticed in percentage of CD45⁺ cells that also expressed CD3. This suggests that the difference seen with both forms of ntEC is due to an acceleration of CD3 recovery rather than a skew in CD45 subpopulations.

E. CD3⁺/CD4⁺ and CD3⁺/CD8⁺ Cell Recovery

The number of CD3⁺ cells that expressed either CD4 or CD8 was increased following ntEC-treatment (FIG. 7). As with other populations, recovery following treatment with ntECs transduced with both E4ORF1 and BMP4 was numerically greater than that with ntECs transduced with only E4ORF1.

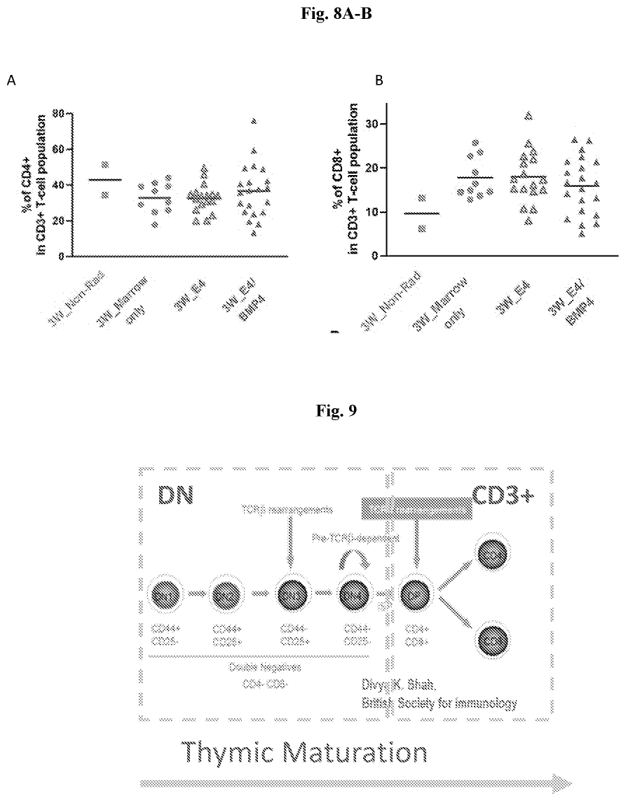

Relative representation of these two populations (the percentage each represents of total CD3⁺ cells) was altered in irradiated animals with a relative increase in CD8 cells and a corresponding decrease in CD4 cells (FIG. 8). The average relative contribution of both populations in animals receiving ntECs transduced with both E4ORF1 and BMP4 tended to be more similar to non-irradiated animals than either animals not receiving ntECs or those animals receiving ntECs transduced with E4ORF1 alone.

### F. Thymic Maturation Markers

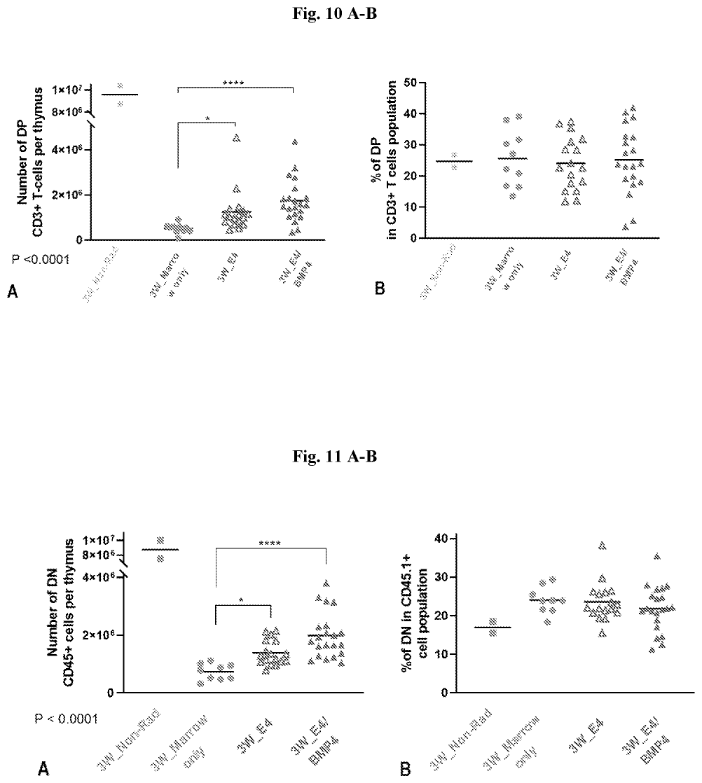

The development of T-lymphocytes within the thymus follows a well-recognized pattern of expression of surface markers (FIG. 9). These markers were applied to allow assessment of the effect of ntEC-treatment on T-lymphocyte development, as described below.

### G. Recovery of CD3 Cells Expressing CD4 and CD8 (Double Positive Cells)

The number of double-positive (DP) cells was increased by treatment with both forms of ntEC though, as with other parameters, the improvement over bone marrow alone was numerically greater for ntECs transduced with both E4ORF land BMP4 than those transduced with E4ORF1 alone (FIG. 10A). The relative number of double-positive cells (as a percentage of all CD3-positive cells) was the same in all groups suggesting that ntEC treatment enhances but does not skew thymic T-cell recovery (FIG. 10B).

### H. Recovery of CD3 Cells Negative for CD4 & CD8 (Double-Negative Cells)

The number of double-negative (DN) cells was increased by treatment with both forms of ntEC though, as with other parameters, the improvement over bone marrow alone was numerically greater for ntECs transduced with both E4ORF land BMP4 than those transduced with E4ORF1 alone (FIG. 11). The mean relative number of DN cells (as a percentage of all CD3-positive cells) was slightly lower (closer to normal) in animals treated with ntECs transduced with both E4ORF land BMP4 (FIG. 11B).

As shown in FIG. 9, double-negative CD3⁺ DN cells can be further sub-divided into 4 cell types based on their expression of CD25, an α-chain of the interleukin 2 (IL-2) receptor, and CD44, an adhesive molecule that participates in a wide variety of cellular functions such as lymphocyte activation, recirculation homing, and hematopoiesis. These four types are designated DN1, DN2, DN3, and DN4 with higher numbers indicating cells that are later in T-cell development.

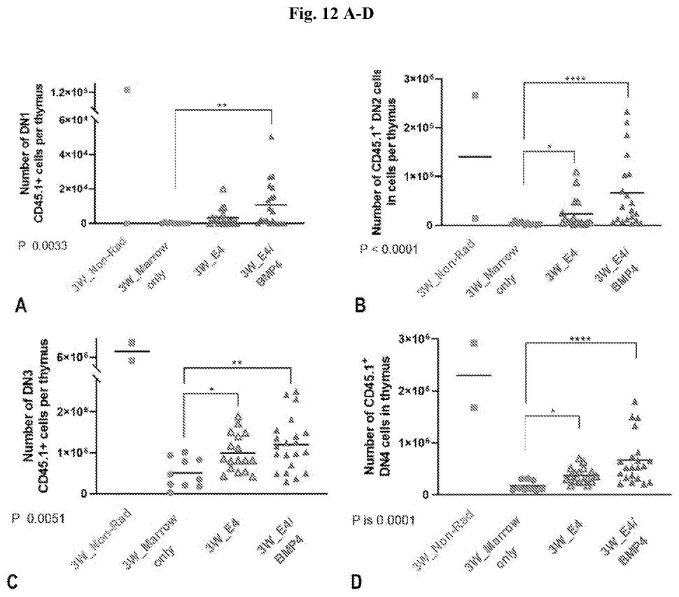

As shown in FIG. 12, the number of each DN subpopulation was greater in animals treated with either ntECs transduced with E4ORF1 alone or ntECs transduced with both E4ORF land BMP4. As with other populations, animals treated with ntECs transduced with both E4ORF1 and BMP4 exhibited numerically greater numbers than those treated with ntECs transduced with E4ORF1 alone though this difference was not statistically significant for any DN subpopulation.

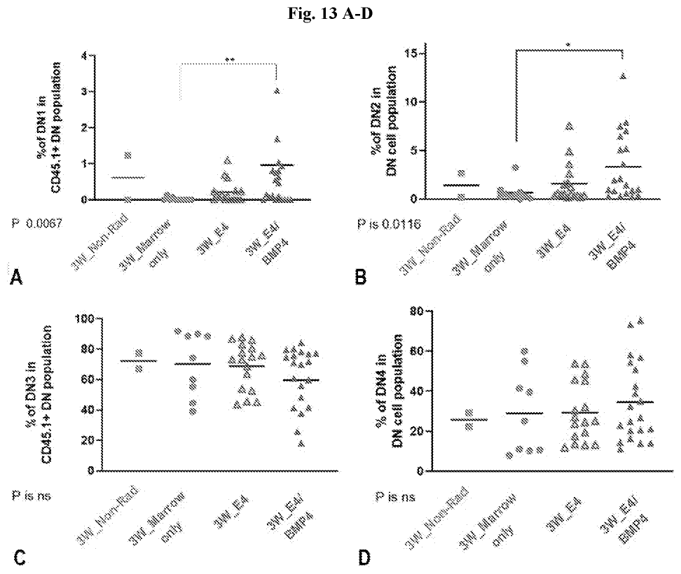

As shown in FIG. 13, the relative amount of DN1, DN2, and DN4 cells/subpopulation was greater in animals treated with either ntECs transduced with E4ORF1 alone or ntECs transduced with both BMP4 and E4ORF1. The percentage of DN3 cells showed a corresponding decrease. The largest relative difference was in the earliest and most rare subpopulations (DN1 and DN2) for which the increase in animals receiving ntECs transduced with both E4ORF land BMP4 was statistically significant.

These data show a consistent pattern in which repopulation of thymic T cells is considerably improved in animals treated with ntECs and in which animals receiving ntECs transduced with both BMP4 and E4ORF1 show greater benefit than those receiving ntECs transduced with E4ORF1 alone. This increased recovery is evident from the earliest T-cell precursor cells assessed (DN1) through to the most mature single positive CD3/CD4 and CD4/CD8 cells.

### I. Thymic Stromal Cell Recovery

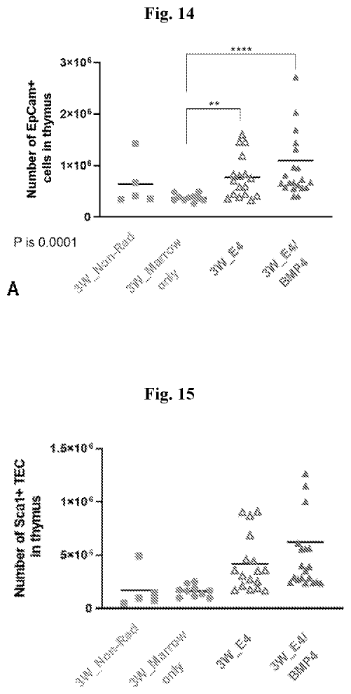

Thymic epithelial cells are characterized by expression of EpCAM and absence of CD45. Out results showed that, as for thymic T-cell populations, animals receiving co-infusion of ntECs exhibited greater numbers of EpCAM+ cells than animals receiving bone marrow alone (FIG. 14). Interestingly, while the average number of such cells in animals receiving bone marrow alone was lower than that of uninjured animals, the number in ntEC-treated animals equaled (E4ORF1 alone) or exceeded (E4+BMP4) that of uninjured animals (FIG. 14). This suggests an overshoot phenomenon though this is largely due to a small number of animals with very high numbers of EpCAM-positive cells (FIG. 14). Data on recovery of various subsets of thymic stromal cells is provided below.

### J. Thymic Epithelial Progenitor Cell (TEPC) Recovery

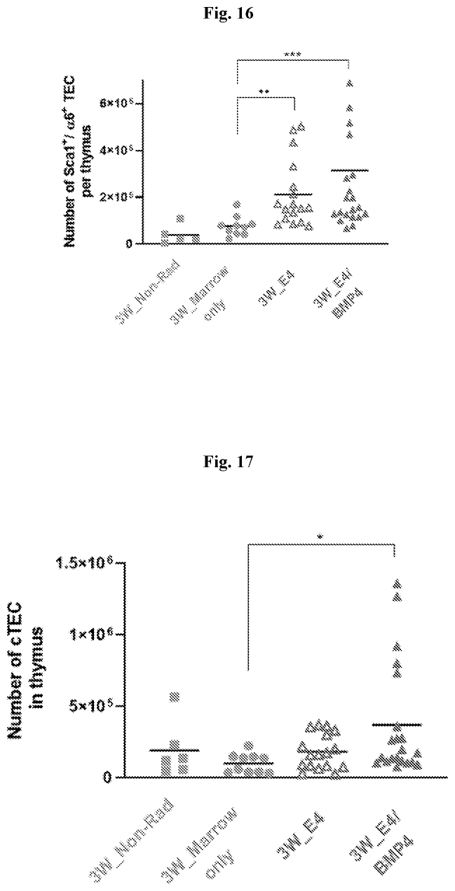

The analysis of Sca1⁺ (FIG. 15) and Sca1⁺/α6⁺ (FIG. 16) thymic epithelial progenitors showed that both populations were increased following treatment with non-thymic endothelial cells and that the effect was magnified with non-thymic endothelial cells transduced with BMP4 (E4ORF land E4/BMP4 respectively).

K. Cortical Thymic Epithelial Cell (cTEC) Recovery

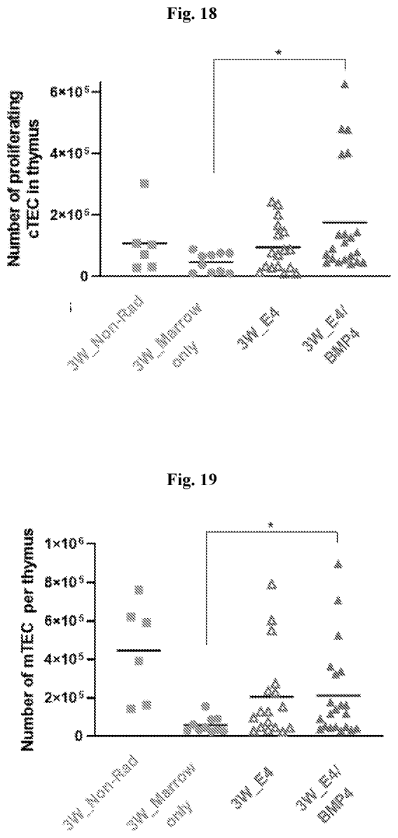

The analysis of mature cortical thymic epithelial cells (cTEC, FIG. 17) showed significantly more cells in the animal group treated with E4/BMP4 in contrast to bone marrow-only group. The analysis of proliferating Ki67+ cTEC showed similar trend (FIG. 18).

L. Medullary Thymic Epithelial Cell (mTEC) Recovery

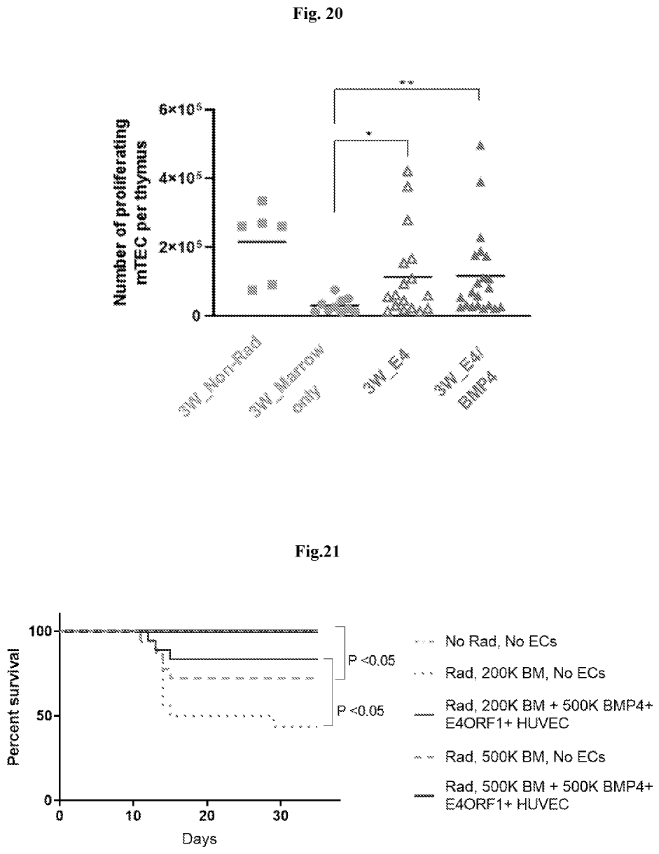

The analysis of mature cortical thymic epithelial cells (mTEC, FIG. 19) showed significantly more cells in the animal group treated with E4/BMP4 in contrast to bone marrow-only group. The analysis of proliferating Ki67+ mTEC showed similar trend (FIG. 20).

The results of the above studies are further summarized in Table 5, below, which shows (in a relative form) the ability of HUVECs, E4ORF1+ HUVECs, BMP4+ HUVECs, or E4ORF1+ HUVECs to accelerate recovery of the listed cell type(s) in the thymus following sublethal irradiation (see “treatment outcome” column). The “+” symbols provide an approximate representation of the cell numbers, with more “+” symbols representing greater cell numbers. For the actual cell numbers please refer to the corresponding data figures.

TABLE 5

| Treatment Outcome | | | | | | |
|---|---|---|---|---|---|---|
| (number of thymic | | | | | | |
| cells following | No | | | | | |
| recovery from | ECs | | | | BMP4 + | |
| irradiation, by | (BM | | E4ORF1 + | BMP4 + | E4ORF1 + | Data |
| cell type) | only) | HUVEC | HUVEC | HUVEC | HUVEC | Subsection |
| Recellularization | + | * | ++ | * | ++++ | A |
| (total thymic | | | | | | |
| cells) | | | | | | |

Hematopoietic Lineage Cells / Thymocytes / T⁻cells & T⁻Cell Progenitors

| | | | | | | |
|---|---|---|---|---|---|---|
| CD45⁺ | + | * | ++ | * | ++++ | C |
| CD3⁺ | + | * | ++ | * | ++++ | D |
| CD3⁺ CD4⁺ | + | * | ++ | * | ++++ | E |
| CD3⁺ CD8⁺ | + | * | ++ | * | ++++ | E |
| CD3⁺ CD4⁺ CD8⁺ | + | * | ++ | * | ++++ | G |
| (DP) | | | | | | |
| CD3⁺ CD4⁻ CD8⁻ | + | * | ++ | * | ++++ | H |
| (DN) | | | | | | |

Thymic Stromal / Epithelial Cells

| | | | | | | |
|---|---|---|---|---|---|---|
| CD45⁻ EpCAM⁺ | + | * | ++ | * | ++++ | I |
| CD45⁻ EpCAM⁺ | + | * | ++ | * | ++++ | J |
| Scal+ (TEPC) | | | | | | |
| CD45⁻ EpCAM⁺ | + | * | ++ | * | ++++ | J |
| Scal+ α6+ | | | | | | |
| (TEPC) | | | | | | |
| CD45⁻ EpCAM⁺ | + | * | ++ | * | ++++ | K |
| (cTEC) | | | | | | |
| CD45− EpCAM⁺ | + | * | ++ | * | ++++ | K |
| Ki67⁺ (cTEC)− | | | | | | |
| proliferating | | | | | | |
| CD45⁻ EpCAM⁺ | + | * | +++ | * | ++++ | L |
| (mTEC) | | | | | | |
| CD45⁻ EpCAM⁺ | + | * | +++ | * | ++++ | L |
| Ki67⁺ (mTEC) | | | | | | |
| proliferating | | | | | | |

* HUVECs and BMP4 + HUVECs (without E4ORF1) could not be grown in sufficient number to use in this in vivo study (see FIG. 1).

### Example 6

### BMP4+ E4ORF1+ ntECs Enhance Survival Following Total Body Irradiation & Bone Marrow Transplant

BMP4+ and E4ORF1+ ntECs were generated essentially as described in Example 1. C57B16 mice were myeloablated with 1,000 cG (a lethal dose) of total body irradiation (TBI) and dosed with either 200,000 or 500,000 whole bone marrow (WBM) cells from syngeneic mice. Some animal cohorts (labelled “+EC” in FIG. 21) were also dosed with BMP4+E4ORF1+ HUVECs (500,000 cells on days 1, 3 and 5) post-TBI.

Survival was assessed at the time points indicated in FIG. 21. As shown in FIG. 5 animals treated with BMP4+E4ORF1+ HUVECs (referred to in FIG. 5 as BMP4 ntECs) and the lower dose of WBM exhibited reduced mortality as compared to animals not treated with ntECs (mortality was reduced by 64%, P<0.05). Dramatically, in animals treated with BMP4+E4ORF1+ ntECs and the higher dose of WBM, mortality was eliminated (survival was 100%, P<0.05). FIG. 21. Thus, at both WBM doses, treatment with BMP4+E4ORF1+ ntECs resulted in a dramatic and statistically significant increase in survival.

The results of the above studies are further summarized in Table 6, below, which summarizes in a relative form the effect of BMP4+E4ORF1+ ntECs on survival following exposure to a lethal dose of radiation. The “+” symbols provide an approximate representation of the survival rate, with more “+” symbols representing greater survival. For the survival numbers please refer to the corresponding data figures. It should be noted that HUVECs and BMP4+ HUVECs (without E4ORF1) could not be grown in sufficient number to use in this in vivo study (see FIG. 1).

TABLE 6

| | No ECs | BMP4+ E4ORF1+ |
|---|---|---|
| Treatment Outcome | (BM alone) | HUVECs |
| Survival following lethal | ++ | ++++ |
| irradiation and transplant with | | |
| suboptimal (200k) bone marrow | | |
| dose | | |
| Survival following lethal | ++ | ++++ |
| irradiation and transplant with | | |
| increased (500k) bone marrow | | |
| dose | | |

### Example 7

### A Human Clinical Trial of BMP4+E4ORF1+ ntECs

A phase 1, open-label, non-randomized, multi-center, multi dose escalation prospective study of BMP4+E4ORF1+ ntECs is performed in adult human subjects with hematologic malignancies undergoing myeloablative conditioning (MAC) and matched related or unrelated donor (MRD or MUD) allogeneic hematopoietic cell transplantation (HCT). This study evaluates the safety and preliminary efficacy of treatment with BMP4+E4ORF1+ ntECs after standard-of-care adult allogeneic HCT.

ntECS from different tissue sources are used (HUVECs and ECs from adipose tissue, skin, lung, heart, kidney and/or bone marrow). BMP4+E4ORF1+ ntECs are produced as described in Example 1.

Enrolled subjects undergo planned HCT as per institutional standards with one of the following MAC regimens—as chosen by the treating oncologist:

total body irradiation (>1000 cGY) (TBI) and cyclophosphamide (120 mg/kg) (Cy/TBI);

etoposide 120 mg/kg and TBI (>1000 cGY), busulfan (16 mg/kg oral or 12.8 mg/kg IV) and cyclophosphamide (120 mg/kg) (Bu/Cy);

busulfan (16 mg/kg PO or 12.8 mg/kg IV) and fludarabine (120-180 mg/m2) (Bu/Flu).

Alternative MAC regimens other than described above are used if and as approved by the study's Medical Monitor. Post-transplant immunosuppression and supportive care is administered per institutional guidelines.

BMP4+E4ORF1+ ntECs are administered intravenously 2 hours after completion of allogeneic (MRD or MUD) HCT infusion on Day 0 upon observation that no acute reaction related to infusion of stem cells has occurred. If acute infusion related reaction occurs during HCT infusion, then the reaction is treated and the subject will not proceed with the trial.

BMP4+E4ORF1+ ntECs are administered in a dose escalation manner, comprising four cohorts (each of at least 6 patients) as shown in Table 7, below.

TABLE 7

| | |
|---|---|
| Cohort 1 | 5 × 10⁶ cells per kilogram subject weight administered on |
| | days 0, 3 and 5 |
| Cohort 2 | 10 × 10⁶ cells per kilogram subject weight administered on |
| | days 0, 3 and 5 |
| Cohort 3 | 20 × 10⁶ cells per kilogram subject weight administered on |
| | days 0, 3 and 5 |
| Cohort 4 | Cell dose displaying best result from cohorts 1-3 is tested as |
| | a single administration (as opposed to the three |
| | administrations in cohorts 1-3). |

The present invention is further described by the following claims.

We claim:

1. A method of enhancing thymic regeneration, the method comprising administering an effective amount of a therapeutic composition comprising engineered non-thymic endothelial cells (ntECs) to a subject in need of thymic regeneration, wherein the engineered ntECs are either E4ORF1+ or BMP4+E4ORF1+, thereby enhancing thymic regeneration in the subject.

2. The method of claim 1, wherein the thymic regeneration comprises recovery of at least one cell type from among CD45− thymic stromal cells and CD45+ T cells.

3. The method of claim 1, wherein the thymic regeneration comprises recovery of both CD45− thymic stromal cells and CD45+ T cells.

4. The method of claim 2 or claim 3, wherein the CD45− thymic stromal cells are selected from the group consisting of thymic epithelial progenitors (TEPCs), cortical thymic epithelial cells (cTECs), and medullary thymic epithelial cells (mTECs).

5. The method of claim 2 or claim 3, wherein the CD45+ T cells are selected from the group consisting of CD3+ T cells, CD4+ T cells, CD8+ T cells, double-positive T cells (DP), double-negative T cells (DN), double-negative type 1 (DN1) T cells, double-negative type 2 (DN2) T cells, double-negative type 3 (DN3) T cells and double-negative type 4 (DN4) T cells.

6. The method of any of the preceding claims, wherein the ntECs are selected from the group consisting of: umbilical vein endothelial cells (UVECs), adipose endothelial cells, skin endothelial cells, lung endothelial cells, heart endothelial cells, kidney endothelial cells and bone marrow endothelial cells.

7. The method of any of the preceding claims, wherein the subject is a human.

8. The method of any of the preceding claims, wherein the ntECs are human umbilical vein endothelial cells (HUVECs).

9. The method of any of the preceding claims, wherein the ntECs are autologous to the subject.

10. The method any of claims 1-8, wherein the ntECs are allogeneic to the subject.

11. The method of any of the preceding claims, wherein the ntECs are MHC/HLA-matched to the subject.

12. The method of any of the preceding claims, wherein the subject has previously been treated with chemotherapy, radiation therapy, a pre-transplantation conditioning regimen, or a myeloablative conditioning regimen.

13. The method of any of the preceding claims, wherein the subject has an immunodeficiency.

14. The method of claim 13, wherein the subject has an HIV infection.

15. The method of any of the preceding claims, wherein the subject has an ageing-related deficiency in thymic tissue mass, thymic function, or T-cell production.

16. The method of any of the preceding claims, wherein the engineered ntECs are administered to the subject by IV infusion.

17. The method of any of the preceding claims, wherein the engineered ntECs are administered to the subject in multiple IV infusions over the course of several days or weeks.

18. The method of any of the preceding claims, further comprising administering to the subject a therapeutic composition comprising hematopoietic stem cells (HSCs).

19. The method of claim 18, wherein engineered ntECs and the HSCs are administered concurrently.

20. The method of claim 18, wherein engineered ntECs and the HSCs are administered to the subject in the same IV infusion.

21. The method of any of the preceding claims, further comprising an initial step of genetically modifying ntECs by transducing or transfecting the ntECs ex vivo with a nucleic acid molecule encoding E4ORF1 and optionally a nucleic acid molecule encoding BMP4, prior to administering the ntECs to the subject.

22. The method of any of the preceding claims, further comprising an initial step of genetically modifying autologous ntECs from the subject by transducing or transfecting the ntECs ex vivo with a nucleic acid molecule encoding E4ORF1 and optionally a nucleic acid molecule encoding BMP4, prior to administering the autologous ntECs to the subject.

23. An isolated population of engineered BMP4+E4ORF1+ non-thymic endothelial cells (ntECs).

24. The population of engineered ntECs of claim 23, wherein the population is a substantially pure population.

25. The population of engineered ntECs of claim 23, wherein the ntECs comprise a recombinant nucleic acid molecule that comprises a nucleotide sequence that encodes BMP4.

26. The population of ntECs of claim 25, wherein the nucleotide sequence that encodes BMP4 is operatively linked to a heterologous promoter.

27. The population of ntECs of claim 25, wherein the ntECs comprise a recombinant nucleic acid molecule that comprises a nucleotide sequence that encodes E4ORF1.

28. The population of ntECs of claim 27, wherein the nucleotide sequence that encodes E4ORF1 is operatively linked to a heterologous promoter.

29. The population of ntECs of any of claims 25-28, wherein the recombinant nucleic acid molecule is an expression vector.

30. The population of ntECs of claim 29, wherein the expression vector is a viral vector.

31. The population of ntECs of claim 29, wherein the expression vector is a lentiviral vector.

32. The population of ntECs of claim 29, wherein the expression vector is a retroviral vector.

33. The population of ntECs of any of claims 23-32, wherein the ntECs are selected from the group consisting of: human umbilical vein endothelial cells (HUVECs), adipose endothelial cells, skin endothelial cells, lung endothelial cells, heart endothelial cells, kidney endothelial cells and bone marrow endothelial cells.

34. The population of ntECs of any of claims 23-32, wherein the ntECs are human umbilical vein endothelial cells (HUVECs).

35. A composition comprising a population of ntECs according to any of claims 23-34.

36. A therapeutic composition comprising a population of ntECs according to any of claims 23-34 and a solution suitable for administration to a subject.

37. A therapeutic composition comprising a population of ntECs according to any of claims 23-34 and a biocompatible matrix material.

38. A therapeutic composition according to claim 37, wherein the biocompatible matrix material is a liquid.

39. A therapeutic composition according to claim 37, wherein the biocompatible matrix material is a solid.
